(19) United States
(12) Patent Application Publication
(10) Pub. No.: US 2012/0148559 A1
(43) Pub. Date: Jun. 14, 2012

(54) COMPOSITIONS AND METHOD FOR DEIMMUNIZATION OF PROTEINS

(75) Inventors: George Georgiou, Austin, TX (US); Jason Cantor, Quincy, MA (US); Tae Hyeon Yoo, Kiheung-gu (KR)

(73) Assignee: Board of Regents The University of Texas System, Austin, TX (US)

(21) Appl. No.: 13/307,715

(22) Filed: Nov. 30, 2011

Related U.S. Application Data
(60) Provisional application No. 61/418,761, filed on Dec. 1, 2010.

(51) Int. Cl. A61K 38/50; C07H 21/04; C12N 5/10; C12N 15/63; C12Q 1/68; C12N 9/78

(52) U.S. Cl. 424/94.6; 435/6.18; 435/227; 435/325; 435/348; 435/349; 435/350; 435/352; 435/353; 435/354; 435/363; 435/364; 435/366; 435/369; 435/371; 435/320.1; 536/23.2

(57) ABSTRACT

The invention provides deimmunized mutant proteins having reduced immunogenicity while exhibiting substantially the same or greater biological activity as the proteins of interst from which they are derived, as exemplified by mutant L-asparaginase that comprises amino acid substitutions compared to wild type L-asparaginase. The invention further provides methods for screening mutant deimmunized proteins that have substantially the same or greater biological activity as a protein of interest, and methods for reducing immunogenicity, without substantially reducing biological activity, of a protein of interest.

The invention's compositions and methods are useful in, for example, therapeutic applications by minimizing adverse immune responses by the host mammalian subjects to the protein of interest. Thus, the invention further provides methods for treating disease comprising administering to a subject a therapeutically effective amount of a pharmaceutical composition comprising at least one of the mutant deimmunized proteins produced by the invention's methods.

### PRIORITY STATEMENT

This application claims priority to co-pending U.S. provisional Application Ser. No. 61/418,761, filed Dec. 1, 2010, which is herein incorporated by reference in its entirety for all purposes.

### STATEMENT OF GOVERNMENT INTEREST

This invention was made with government support under grant CA139059 awarded by the National Institutes for Health (NIH). The government has certain rights in the invention.

### FIELD OF INVENTION

The invention provides deimmunized mutant proteins having reduced immunogenicity while exhibiting substantially the same or greater biological activity as the proteins of interest from which they are derived, as exemplified by mutant L-asparaginase that comprises amino acid substitutions compared to wild type L-asparaginase. The invention further provides methods for screening mutant proteins (such as deimmunized proteins) that have substantially the same or greater biological activity as a protein of interest, and methods for reducing immunogenicity, without substantially reducing biological activity, of a protein of interest.

The invention's compositions and methods are useful in, for example, therapeutic applications by minimizing adverse immune responses by the host mammalian subjects to the protein of interest. The invention is also useful for isolating variants of a human enzyme that displays a novel and/or improved catalytic activity towards the degradation of an amino acid. The invention further provides methods for treating disease comprising administering to a subject a therapeutically effective amount of a pharmaceutical composition comprising at least one of the mutant deimmunized proteins produced by the invention's methods.

### BACKGROUND

A variety of genetic and acquired human diseases can be treated by the systemic administration of proteins, such as enzymes catalyzing the depletion of metabolites that contribute to pathological states. Recombinant human enzymes are used extensively as replacement therapy for lysosomal storage disorders such as Gaucher's, Fabry's, and Pompe disease (1). However, there are many diseases for which a human enzyme displaying the requisite catalytic and pharmacological properties for clinical use is unavailable. Therefore, heterologous enzymes, primarily of bacterial origin, have been evaluated for the treatment of a variety of disorders including phenylketonuria (PKU) (2), gout (3), and a number of cancers that are sensitive to enzyme-mediated, systemic depletion of amino acids. Examples of the latter include a large fraction of hepatocellular carcinomas and metastatic melanomas that become apoptotic under conditions where the non-essential amino acid L-arginine in serum is depleted (4), central nervous system cancers that respond to L-Met deprivation (5), and acute lymphoblastic leukemia (ALL) for which enzyme-mediated L-asparagine depletion is a critical step in the current clinical treatment approach (6-8).

A major impediment in the therapeutic application of heterologous enzymes is their immunogenicity, which results in the generation of anti-enzyme antibodies that in turn mediate a variety of adverse effects including hypersensitivity reactions, anaphylactic shock, and the inactivation and clearance of the enzyme itself (9). For example, immunogenicity is a major problem in enzyme therapy for cancer and other indications. In particular in ALL up to 60% of the patients ultimately developed antibodies to L-Asparaginase that lead to reduced efficacy and discontinuation of treatment. Masking of immunogenic epitopes via covalent modification with polyethylene glycol (PEG) can reduce protein immunogenicity (10), but eventually, antigen-specific and even PEG-specific, antibodies that contribute to therapeutic neutralization may be elicited towards PEGylated proteins (11, 12).

Protein immunogenicity can be ameliorated by mutating sequences likely to be recognized by the naïve antibody repertoire (B-cell epitopes) or sequences that are bound by the major histocompatibility complex (MHC)-II and thus can elicit T cell-dependent (Td) immune responses. However, the prior art has been faced with difficulty in the identification and removal of B-cell epitopes given their conformational nature, which is further complicated by the prior art's incomplete knowledge of the naïve antibody repertoire, and how they vary across different human populations.

There is extensive evidence from animal models, in vitro experiments, and early stage clinical studies, that the disruption of T-cell epitopes can reduce antibody responses in some therapeutic proteins (13, 14). T-cell receptors on CD4+ T-cells recognize antigenic peptides (typically 13-25mers) presented in complex with MHC-II molecules on the surface of antigen presenting cells (APCs). The MHC-II binding groove contains four well-defined pockets that accommodate the side chains of the P1, P4, P6, and P9 residues within a core 9mer region of the T-cell epitope and these key residues largely determine binding affinity and specificity (15). Although the MHC-II locus is highly polymorphic, assays using APCs from volunteers representative of the major MHC-II haplotypes in human populations have been deployed successfully to identify T-cell epitopes that contribute to protein immunogenicity in a large fraction of patients. Alternatively, a plethora of in silico methods (16) have been developed over the past several years for the prediction of sequences that bind to various MHC-II alleles.

Heterologous enzymes that have not undergone immunological tolerance induction typically contain multiple T-cell epitopes. Therefore, the removal of putative T cell epitopes necessitates extensive alteration of the polypeptide sequence in a manner that does not affect protein function. However, the introduction of multiple amino acid substitutions that disrupt MHC II binding but do not affect catalytic activity represents a significant challenge in the prior art. This is particularly problematic when deimmunization requires the replacement of amino acids that are phylogenetically conserved and consequently, substitutions at these positions could impact protein stability or catalytic efficiency.

The removal of T-cell epitopes by mutagenesis has been used to reduce the immunogenicity of humanized and chimeric antibodies (17-19). However, whereas these proteins contain, at most, a few relatively short potentially immunogenic sequences, heterologous enzymes that have not undergone immunological tolerance induction typically contain multiple T-cell epitopes, the removal of which thus necessitates extensive alteration of the polypeptide sequence in a manner that does not affect protein function. Further, enzyme catalysis is dictated not only by the active site residues, but also on a network of amino acids distributed throughout the protein (20). For this reason, the introduction of multiple amino acid substitutions that disrupt MHC-II binding but do not affect catalytic activity represents a significant challenge. This is particularly problematic when deimmunization requires the replacement of amino acids that are phylogenetically conserved and consequently, substitutions at these positions could impact protein stability or catalytic efficiency. Rational approaches for the incorporation of deimmunizing mutations into heterologous enzymes have previously proven effective (14, 21), however the tolerable mutations necessary to reduce immunogenicity while concomitantly maintaining enzyme functionality may not always be readily determined by these means.

Thus, there remains a need for alternative methods and compositions to screen functional proteins (e.g., enzymes) that retain or increase their biological activity, their biological half-life, and/or their biochemical stability, in combination with attenuated immunogenicity.

### SUMMARY OF THE INVENTION

The invention provides a mutant L-asparaginase that A) comprises 8 amino acid substitutions that correspond to substitution of wild type L-asparaginase SEQ ID NO:03 1) methionine at position 115 with valine (M115V), 2) serine at position 118 with proline (S118P), 3) serine at position 120 with arginine (S120R), 4) alanine at position 123 with proline (A123P), 5) isoleucine at position 215 with valine (1215V), 6) asparagine at position 219 with glycine (N219G), 7) glutamine at position 307 with threonine (Q307T), and 8) glutamine at position 312 with asparagine (Q312N), B) has the same or greater enzyme activity as wild type L-asparaginase SEQ ID NO:03, and C) has reduced immunogenicity compared to wild type L-asparaginase SEQ ID NO:03. In one embodiment, the mutant L-asparaginase has the same or greater stability of enzyme activity in serum as wild type L-asparaginase SEQ ID NO:03. In a particular embodiment, the mutant L-asparaginase comprises SEQ ID NO:01.

The invention also provides a pharmaceutical composition comprising any one or more of the mutant L-asparaginase described herein, and a carrier.

Also provided is recombinant nucleotide sequence encoding any one or more of the mutant L-asparaginase described herein. In one embodiment, the nucleotide sequence comprises SEQ ID NO:02.

The invention also provides an expression vector that comprises a nucleotide sequence encoding any one or more of the mutant L-asparaginase described herein.

The invention further provides a transgenic cell comprising one or more of the expression vectors described herein, including vectors and that that comprise a nucleotide sequence encoding any one or more of the mutant L-asparaginase described herein.

In one embodiment, the invention provides a method for identifying a mutant deimmunized protein that has the same or greater biological activity as a protein of interest, comprising A) providing i) a first plurality of first expression vectors, wherein each expression vector comprises in operable combination 1) a first nucleotide sequence encoding a mutant of a protein of interest, wherein the protein of interest comprises one or more epitope sequence, and wherein the mutant protein contains one or more mutations in one or more of the epitope sequence, 2) a reporter nucleotide sequence, and 3) a promoter, ii) a transgenic cell that lacks expression of a biologically active the protein of interest, B) transfecting the transgenic cell with the first plurality of first expression vectors to produce a first plurality of populations of transfected transgenic cells, wherein each population of the first plurality of populations of transfected transgenic cells comprises one of the first expression vectors, C) culturing the first plurality of populations of transfected transgenic cells under conditions for expression of the first nucleotide sequence and of the reporter nucleotide sequence, D) detecting expression of the reporter nucleotide sequence in the first plurality of populations of transfected transgenic cells, wherein the level of the expression of the reporter nucleotide sequence correlates with the level of biological activity of the mutant protein that is encoded by the operably linked first nucleotide sequence, and E) determining the immunogenicity in the first plurality of populations of transfected transgenic cells of the expressed mutant protein, wherein i) detecting the same or greater biological activity of the mutant protein compared to the protein of interest, and ii) detecting reduced immunogenicity of the mutant protein compared to the protein of interest, identifies the mutant protein as a deimmunized protein that has the same or greater biological activity as the protein of interest. In one embodiment, the method further comprises F) providing i) a second plurality of second expression vectors, wherein each expression vector of the second plurality of expression vectors comprises in operable combination 1) a second nucleotide sequence encoding a variant of the identified mutant protein, wherein the variant protein contains additional one or more mutations in the one or more epitope sequence of the identified mutant protein, 2) a reporter nucleotide sequence, and 3) a promoter, ii) a transgenic cell that lacks expression of a biologically active the protein of interest, G) transfecting the transgenic cell with the second plurality of expression vectors to produce a second plurality of populations of transfected transgenic cells, wherein each population of the second plurality of populations of transfected transgenic cells comprises one of the second expression vectors, H) culturing the second plurality of populations of transfected transgenic cells under conditions for expression of the second nucleotide sequence and the reporter nucleotide sequence, I) detecting expression of the reporter nucleotide sequence in the second plurality of populations of transfected transgenic cells, wherein the level of the expression of the reporter nucleotide sequence correlates with the level of biological activity of the variant protein that is encoded by the operably linked second nucleotide sequence, and J) determining the immunogenicity in the plurality of populations of transfected transgenic cells of the expressed variant protein, wherein i) detecting the same or greater biological activity of the variant protein compared to the protein of interest, and ii) detecting reduced immunogenicity of the variant protein compared to the protein of interest, identifies the variant protein as a deimmunized protein that has the same or greater biological activity as the protein of interest. In a particular embodiment, the method further comprises detecting the stability of the biological activity of the mutant protein. In an alternative embodiment, the method further comprises purifying the identified mutant deimmunized protein.

In another embodiment, the method further comprises detecting one or more mutation in the epitope sequence of the purified mutant deimmunized protein. In a particular embodiment, the method further comprises determining immunogenicity of the purified mutant deimmunized protein. In a further embodiment, the method the protein of interest is an enzyme, and the transgenic cell further lacks expression of a product produced by the enzyme activity of a wild type of the enzyme. In a more particular embodiment, the reporter nucleotide sequence comprises a gene encoding a fluorescent protein. In yet a further embodiment, the protein of interest is selected from the group of enzyme of interest and binding protein of interest. In a further embodiment, the enzyme of interest is an amino acid degrading enzyme. In an alternative embodiment, the amino acid degrading enzyme comprises L-Asparaginase. In another embodiment, the reduced immunogenicity comprises from 1 to 10,000 fold lower immunogenicity of the mutant protein compared to immunogenicity of the protein of interest.

The invention also provides a pharmaceutical composition comprising the mutant protein identified by any one or more of the methods described herein, and a carrier.

The invention additionally provides a method for reducing immunogenicity of a protein of interest without reducing biological activity of the protein of interest, comprising a) identifying a mutant of the protein of interest using any one or more of the methods herein, b) determining the amino acid sequence of one or more the epitope sequence in the identified mutant protein, and c) producing a variant protein of interest that contains the determined epitope sequence.

In a separate embodiment, the invention provides a method for identifying a mutant mammalian enzyme that has a desired level of catalytic activity for degradation of an amino acid, comprising: A) providing i) a plurality of expression vectors, wherein each expression vector comprises in operable combination 1) a nucleotide sequence encoding a mutant of said mammalian enzyme, 2) a reporter nucleotide sequence, and 3) a promoter, and ii) a transgenic cell that lacks expression of enzymes having the catalytic activity, B) transfecting the transgenic cell with the plurality of expression vectors to produce a plurality of populations of transfected transgenic cells, wherein each population of transfected transgenic cells comprises one of the expression vectors, C) culturing the plurality of populations of transfected transgenic cells under conditions for expression of the nucleotide sequence encoding said mutant and for expression of the reporter nucleotide sequence, D) detecting expression of the reporter nucleotide sequence in one or more of the plurality of populations of transfected transgenic cells, wherein the level of expression of the reporter nucleotide sequence correlates with the level of said catalytic activity of the mammalian mutant enzyme that is encoded by the operably linked nucleotide sequence, and wherein said detecting identifies said mutant mammalian enzyme as having a desired level of said catalytic activity. In a particular embodiment, the method further comprises determining the level of expression of the reporter nucleotide sequence. In another embodiment, the mammalian enzyme is a human enzyme.

Also provided herein is a pharmaceutical composition comprising the variant protein of interest produced by any one or more of the methods herein, and a carrier.

The invention also provides a method for treating disease comprising administering to a mammalian subject in need thereof a therapeutically effective amount of a pharmaceutical composition comprising at least one protein selected from a) any one or more of the mutant L-asparaginase described herein, b) any one or more mutant deimmunized protein identified by any one or more of the methods herein, and c) any one or more variant protein of interest produced by any one or more of the methods herein. In one embodiment, the protein is heterologous to the subject. In another embodiment, the protein is selected from the group of enzyme and binding protein.

### BRIEF DESCRIPTION OF THE DRAWINGS

FIG. 1. Deimmunization by combinatorial T-cell epitope removal using the invention's neutral drift. (a) Methodology for combinatorial T-cell epitope removal by the invention's neutral drift and subsequent evaluation of isolated variants using HLA-transgenic mice. (b) High-throughput neutral drift FACS screen for L-asparaginase. μ: geometric mean fluorescence.

FIG. 2. Isolation of EcaII neutral drift mutants (a) Residue plasticity of known bacterial type II asparaginases (IPRO04550; n=478) and of EcAII variants exhibiting WT activity isolated by neutral drift (n=20 per library) at the 12 amino acids targeted for mutagenesis. Residue numbers correspond to positions in EcAII. (b) Location of the 8 amino acid mutations differentiating wild type EcAII and 3.1.E2. Images generated by PyMol (55). The location of these mutations in relation to the EcAII active site is shown in FIG. 8.

FIG. 3. T cell activation and antibody responses in HLA-DRB1*0401 transgenic mice: (a) IFN-γ production by lymph node T-cells from HLA-DRB1*0401 transgenic mice immunized with either EcAII (n=10 mice) or 3.1.E2 (n=8 mice) and challenged with overlapping 20-mer peptides as shown. For each mouse, cytokine signals to each peptide were normalized using the cytokine signal generated to whole antigen in order to account for response variability across each sample population. Error bars shown are S.E.M. *: p=0.182; **: p<0.0001: paired Student's t-test, 2-tailed, comparing recall responses. (b) Relative anti-EcAII serum IgG titers induced by EcAII (n=6 mice) and 3.1.E2 (n=6 mice) in HLA-DRB1*0401 transgenic mice. Error bars shown are S.E.M. *: p=0.02: unpaired Student's t-test, 2-tailed, comparing antibody titers.

FIG. 4: Validation of the invention's neutral drift screen for cells expressing EcAII variants with high catalytic activity. (a) Relative GFP signal of a panel of E. coli JC1 cells expressing EcAII variants with different catalytic efficiencies for the hydrolysis of the L-Asn analog AHA (1). The T12A mutant displays no AHA hydrolysis activity above background in this assay. (b) Fluorescence histograms showing 3 round enrichment of JC1 cells expressing EcAII from a mixture containing a 1:10,000 excess of JC1 cells expressing EcAII-T12A. After 3 rounds of sorting, DNA sequencing revealed that 5 of 8 clones selected at random encoded EcAII. μ: geometric mean fluorescence.

FIG. 5: FACS histograms of 4 residue saturation libraries at the anchor positions in the predicted T cell epitope 9-mer peptides M₁₁₅, I₂₁₆, V₃₀₄ (see Main text for nomenclature) by the invention's neutral drift assay. Each library comprised of >10⁷ transformants generated by randomizing the P1, P4, P6 and P9 positions of the respective T cell epitopes using the NNS scheme. (a) M₁₁₅ library (b) I₂₁₆ library (c) V₃₀₄ library. μ: geometric mean fluorescence.

FIG. 6: In serum stability of WT EcAII and 3.1.E2 measured quantitatively by percent remaining activity over time. Error bars shown are for S.D.

FIG. 7: Reducing SDS-PAGE showing the purity of purified WT EcAII and engineered EcAII variants. Lane 1: WT EcAII; Lane 2: 1.1.C4; Lane 3: 2.2.G10; Lane 4: 3.1.E2; Lane 5: M.W. standards

FIG. 8: Location of amino acid mutations of 3.1.E2 in relation to the active site residues of EcAII. (a) Location of the 8 amino acid mutations differentiating wild type EcAII and 3.1.E2. (b) Active site residues of EcAII (2) are depicted in green and numbered. Residues numbered with a prime are located in an adjacent monomer.

FIG. 9: E. coli type II asparaginase. (A) 3.1.E2 (mature sequence) amino acid sequence (SEQ ID NO:01), and DNA sequence encoding it (SEQ ID NO:02). (B) Wild-type (mature sequence) amino acid sequence (SEQ ID NO:03) and DNA sequence encoding it (SEQ ID NO:04). Differences between the wild-type and 3.1.E2 sequences are in bold.

FIG. 10: A. Arginine Deiminase (Mycoplasma arginini) wild-type amino acid sequence UniProt #P23793 (SEQ ID NO:05). B. L-methioninase (Psuedomonas putida) wild-type amino acid sequence UniProt #P13254 (SEQ ID NO:06). C. Phenylalanine ammonia lyase (PAL) (Anabeaena variabilis) wild-type amino acid sequence UniProt #Q3M5Z3 (SEQ ID NO:07). D. Urate Oxidase (Aspergillus flavus) UniProt #Q00511(SEQ ID NO:08). E. Ecotin (E. coli) wild-type amino acid sequence UniProt #P23827 (SEQ ID NO:09). F. glutamine deaminase (Burkholderia xenovorans) wild-type amino acid sequence UniProt #Q145Z3 (SEQ ID NO:10).

FIG. 11: Validation of the invention's screen for cells expressing methioninase. Fluorescence histograms showing 3 round enrichment of BL21(DE3)(ΔilvA, ΔmetA) cells expressing the P. putida methionine-gamma-lyase enzyme (pMGL) from a mixture containing a 1:10,000 excess of cells expressing pET28a plasmid containing the gene for the human cystathionine-gamma-lyase (hCGL) which displays no methioninase activity. After 3 rounds of sorting, DNA sequencing revealed that 6/12 clones selected at random encoded pMGL.

### DEFINITIONS

To facilitate understanding of the invention, a number of terms are defined below.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed using a recombinant DNA molecule.

The term “recombinant mutation” refers to a mutation that is introduced by means of molecular biological techniques. This is in contrast to mutations that occur in nature.

“Protein of interest” “peptide of interest,” “nucleotide sequence of interest,” and “molecule of interest” refer to any peptide sequence, nucleotide sequence, and molecule, respectively, the manipulation of which may be deemed desirable for any reason, by one of ordinary skill in the art, including wild type sequences, heterologous sequences, mutant sequences, etc.

The terms “endogenous” and “wild type” when in reference to a sequence refer to a sequence that is naturally found in the cell into which it is introduced so long as it does not contain some modification relative to the naturally-occurring sequence. The term “heterologous” refers to a sequence that is not endogenous to the cell into which it is introduced.

The term “heterologous” refers to a sequence which is not endogenous to the cell into which it is introduced. For example, a “heterologous” gene refers to a gene that is not in its natural environment (in other words, has been altered by the hand of man). For example, a heterologous gene includes a gene from one species introduced into another species. A heterologous gene also includes a gene native to an organism that has been altered in some way (for example, mutated, added in multiple copies, linked to a non-native promoter or enhancer sequence, etc.). Heterologous genes may comprise gene sequences that comprise cDNA forms of a gene; the cDNA sequences may be expressed in either a sense (to produce mRNA) or anti-sense orientation (to produce an anti-sense RNA transcript that is complementary to the mRNA transcript). Heterologous genes are distinguished from endogenous plant genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (for example, genes expressed in loci where the gene is not normally expressed).

The terms “mutation” and “modification” refer to a deletion, insertion, or substitution. Thus a “mutant” amino acid sequence refers to an amino acid sequence that contains one or more deletion, insertion and/or substitution compared to a reference amino acid sequence. Similarly, a “mutant” nucleotide sequence refers to a protein that contains one or more deletion, insertion and/or substitution compared to a reference nucleotide sequence. Mutants may be produced using methods know in the art, such as site-directed mutagenesis, randomization of one or more nucleotides in a nucleotide sequence encoding the mutant protein (Example 4), etc.

A “deletion” is defined as a change in a nucleic acid sequence or amino acid sequence in which one or more nucleotides or amino acids, respectively, is absent.

An “insertion” or “addition” is that change in a nucleic acid sequence or amino acid sequence that has resulted in the addition of one or more nucleotides or amino acids, respectively.

A “substitution” in a nucleic acid sequence or an amino acid sequence results from the replacement of one or more nucleotides or amino acids, respectively, by a molecule that is a different molecule from the replaced one or more nucleotides or amino acids. For example, a nucleic acid may be replaced by a different nucleic acid as exemplified by replacement of a thymine by a cytosine, adenine, guanine, or uridine. Alternatively, a nucleic acid may be replaced by a modified nucleic acid as exemplified by replacement of a thymine by thymine glycol. Substitution of an amino acid may be conservative or non-conservative. “Conservative substitution” of an amino acid refers to the replacement of that amino acid with another amino acid which has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains that may be conservatively substituted one for the other include phenylalanine, tyrosine, and tryptophan. Cysteine and methionine are sulphur-containing amino acids which may be conservatively substituted one for the other. Also, asparagine may be conservatively substituted for glutamine, and vice versa, since both amino acids are amides of dicarboxylic amino acids. In addition, aspartic acid (aspartate) may be conservatively substituted for glutamic acid (glutamate) as both are acidic, charged (hydrophilic) amino acids. Also, lysine, arginine, and histidine may be conservatively substituted one for the other since each is a basic, charged (hydrophilic) amino acid. “Non-conservative substitution” is a substitution other than a conservative substitution. Guidance in determining which and how many amino acid residues may be substituted, inserted or deleted without abolishing biological and/or immunological activity may be found using computer programs well known in the art, for example, DNAStar™ software.

“Conserved” when referring to an amino acid, nucleotide, amino acid sequence, and/or nucleotide sequence in two molecules refers to 100% identity of the amino acid, nucleotide, amino acid sequence, and/or nucleotide sequence in the two molecules. Conserved sequences may be determined by sequence alignment.

“Correspond” and “corresponding” when in reference to the position of a first amino acid in a first polypeptide sequence as compared to a second amino acid in a second polypeptide sequence means that the positions of the first and second amino acids are aligned when the first and second amino acid sequences are aligned.

A “variant” or “homolog” of a polypeptide sequence of interest or nucleotide sequence of interest, refers to a sequence that has at least 80% identity, including 81%, 82%, 83%, 84%, 85%, 86%, 87%, 88%, 89%, 90%, 91%, 92%, 93%, 94%, 95%, 96%, 97%, 98%, and 99%, identity with the a polypeptide sequence of interest or nucleotide sequence of interest, respectively. Thus, in one embodiment, a homologous sequence refers to a sequence that contains a mutation relative to the sequence of interest. In another embodiment, the homologous nucleotide sequence refers to a sequence that hybridizes under stringent conditions to the nucleotide sequence of interest.

“Stringent conditions” when used in reference to nucleic acid hybridization comprise conditions equivalent to binding or hybridization at 42° C. in a solution of 5×SSPE (43.8 g/l NaCl, 6.9 g/l NaH₂PO₄—H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed. In another embodiment, high stringency conditions comprise conditions equivalent to binding or hybridization at 68° C. in a solution containing 5×SSPE, 1% SDS, 5×Denhardt's reagent and 100 μg/ml denatured salmon sperm DNA followed by washing in a solution containing 0.1×SSPE, and 0.1% SDS at 68° C.

“Identity” when in reference to 2 or more sequences (e.g., DNA, RNA, and/or protein sequences) refers to the degree of similarity the 2 or more sequences, and is generally expressed as a percentage. Identity in amino acid or nucleotide sequences can be determined using Karlin and Altschul's BLAST algorithm (Proc. Natl. Acad. Sci. USA, 1990, 87, 2264-2268; Karlin, S. & Altschul, S F., Proc. Natl. Acad. Sci. USA, 1993, 90, 5873). Programs called BLASTN and BLASTX have been developed using the BLAST algorithm as a base (Altschul, S F. et al., J. Mol. Biol., 1990, 215, 403). When using BLASTN to analyze nucleotide sequences, the parameters can be set at, for example, score=100 and word length=12. In addition, when using BLASTX to analyze amino acid sequences, the parameters can be set at, for example, score=50 and word length=3. When using BLAST and the Gapped BLAST program, the default parameters for each program are used. Specific techniques for these analysis methods are the well-known, e.g., on the website of the National Center for Biotechnology Information

“Vector” and “vehicle” are used interchangeably in reference to nucleic acid molecules that transfer nucleotide sequences from one cell to another. Vectors are exemplified by, but not limited to, plasmids such as bacterial artificial chromosomes (BACs), linear DNA, encapsidated virus, etc. that may be used for expression of a desired sequence.

“Expression vector” as used herein refers to a recombinant DNA molecule containing a desired coding sequence and appropriate nucleic acid sequences necessary for the expression (i.e., transcription and/or translation) of the operably linked coding sequence in a particular host organism. Expression vectors are exemplified by, but not limited to, plasmid, phagemid, shuttle vector, cosmid, virus, chromosome, mitochondrial DNA, plastid DNA, and nucleic acid fragment. Nucleic acid sequences used for expression in prokaryotes include a promoter, optionally an operator sequence, a ribosome binding site and possibly other sequences. Eukaryotic cells are known to utilize promoters, enhancers, and termination and polyadenylation signals.

“Transgenic” cell refers to a cell that contains a transgene, or whose genome has been altered by the introduction of a “transgene.” Transgenic cells may be produced by several methods including the introduction of a “transgene” comprising nucleic acid (usually DNA) sequences into a target cell or integration of the transgene into a chromosome of a target cell by way of human intervention, such as by the methods described herein.

The term “transgene” as used herein refers to any nucleic acid sequence which is introduced into the cell by experimental manipulations. A transgene may be an “endogenous DNA sequence” or a “heterologous DNA sequence” (i.e., “foreign DNA”). The terms “purified,” “isolated,” and grammatical equivalents thereof as used herein, refer to the reduction in the amount of at least one undesirable component (such as cell, protein, nucleic acid sequence, carbohydrate, etc.) from a sample, including a reduction by any numerical percentage of from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100%. Thus purification results in “enrichment,” i.e., an increase in the amount of a desirable component cell, protein, nucleic acid sequence, carbohydrate, etc.) relative to the undesirable component.

“Operable combination” and “operably linked” when in reference to the relationship between nucleic acid sequences and/or amino acid sequences refers to linking the sequences such that they perform their intended function. For example, operably linking a promoter sequence to a nucleotide sequence of interest refers to linking the promoter sequence and the nucleotide sequence of interest in a manner such that the promoter sequence is capable of directing the transcription of the nucleotide sequence of interest and/or the synthesis of a polypeptide encoded by the nucleotide sequence of interest.

The term “specifically binds” and “specific binding” when made in reference to the binding of antibody to a molecule (e.g., peptide) or binding of a cell (e.g., T-cell) to a peptide, refer to an interaction of the antibody or cell with one or more epitopes on the molecule where the interaction is dependent upon the presence of a particular structure on the molecule. For example, if an antibody is specific for epitope “A” on the molecule, then the presence of a protein containing epitope A (or free, unlabeled A) in a reaction containing labeled “A” and the antibody will reduce the amount of labeled A bound to the antibody. In one embodiment, the level of binding of an antibody to a molecule is determined using the “IC50” i.e., “half maximal inhibitory concentration” that refer to the concentration of a substance (e.g., inhibitor, antagonist, etc.) that produces a 50% inhibition of a given biological process, or a component of a process (e.g., an enzyme, antibody, cell, cell receptor, microorganism, etc.). It is commonly used as a measure of an antagonist substance's potency.

A “subject” that may benefit from the invention's methods includes any multicellular animal, preferably a “mammal.” Mammalian subjects include humans, non-human primates, murines, ovines, bovines, ruminants, lagomorphs, porcines, caprines, equines, canines, felines, ayes, etc.). Thus, mammalian subjects are exemplified by mouse, rat, guinea pig, hamster, ferret and chinchilla. The invention's compositions and methods are also useful for a subject “in need of reducing one or more symptoms of a disease includes a subject that exhibits and/or is at risk of exhibiting one or more symptoms of the disease.

For Example, subjects may be “at risk” for disease, i.e., predisposed to contracting and/or expressing one or more symptoms of the disease, based on family history, genetic factors, environmental factors such as exposure to carcinogens, etc. This term includes animal models of the disease and subjects “suffering from disease,” i.e., experiencing one or more symptoms of the disease. It is not intended that the present invention be limited to any particular signs or symptoms, and expressly includes sub-clinical and/or clinical symptoms of full-blown disease. Thus, administering a composition to a subject in need of reducing one or more symptoms of the disease includes prophylactic administration of the composition (i.e., before the disease and/or one or more symptoms of the disease are detectable) and/or therapeutic administration of the composition (i.e., after the disease and/or one or more symptoms of the disease are detectable).

“Biological activity” of a molecule refers to the effect of the molecule on living matter (e.g., on a cell, virus, etc.) and/or components of living matter (e.g., proteins, nucleotide sequences, etc.). Biological activity includes enzyme activity (e.g., galactosidases, insulin, etc.), cell signaling activity (e.g., hormones, neurotransmitters, cytokines, growth factors, etc.), receptor activity (e.g., hormone receptors, neurotransmitter receptors, cytokine receptors, growth factor receptors, ion channel receptors, etc.), ligand binding protein activity (e.g., hemoglobin, lectins, etc.), DNA-binding activity, antibody activity, structural protein function (e.g., collagen, elastin, keratin, etc.), and the like.

“Stability,” “stable” and grammatical equivalents when in reference to biological activity (e.g., enzyme activity) refer to the rate of change, or lack of change, of biological activity over time.

The terms “antigen,” “immunogen,” “antigenic,” “immunogenic,” “antigenically active,” “immunologic,” and “immunologically active” when made in reference to a molecule, refer to any substance that is capable of inducing a specific humoral immune response (including eliciting a soluble antibody response) and/or cell-mediated immune response (including eliciting a cytotoxic T-lymphocyte (CTL) response). To elicit antibody production, in one embodiment, small molecules, or haptens, may be conjugated to keyhole limpet hemocyanin (KLH), bovine serum albumin (BSA) or fused to glutathione-S-transferase (GST).

“Immunogenicity” of a molecule refer to the ability of the molecule to inducing a specific humoral immune response (including eliciting a soluble antibody response) and/or cell-mediated immune response (including eliciting a cytotoxic T-lymphocyte (CTL) response). Methods for determining immunogenicity are known in the art, and disclosed herein such as measuring T-cell responses and/or IgG titers in mice treated with the invention's compositions.

The terms “epitope” and “antigenic determinant” refer to a structure on an antigen that interacts with the binding site of an antibody or T cell receptor as a result of molecular complementarity. An epitope may compete with the intact antigen, from which it is derived, for binding to an antibody. Generally, secreted antibodies and their corresponding membrane-bound forms are capable of recognizing a wide variety of substances as antigens, whereas T cell receptors are capable of recognizing only fragments of proteins which are complexed with MHC molecules on cell surfaces. Antigens recognized by immunoglobulin receptors on B cells are subdivided into three categories: T-cell dependent antigens, type 1 T cell-independent antigens; and type 2 T cell-independent antigens. Also, for example, when a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

“De-immunized” and “deimmunized” when in reference to a molecule of interest (e.g., a mutant protein) as compared to a reference molecule (e.g., a wild-type protein) means that the molecule of interest has a reduced level (including, but not limited to a complete absence) of immunogenicity compared to the reference molecule.

“Enzyme” refers to a protein that catalyzes chemical reactions of other substances without itself being destroyed or altered upon completion of the reactions.

“Amino acid degrading enzyme” refers to an enzyme that catalyzes break down of an amino acid, such as L-asparaginase, arginine deiminase, L-methioninase, phenylalanine ammonia lyase (PAL), urate oxidase, ecotin, and glutamine deaminase.

“Enzyme activity” and grammatical equivalents (e.g., “enzymically active,” “enzymatically active,” etc.) refer to a chemical reaction or process produced by the action of an enzyme on a substrate to produce a product, while the enzyme is not consumed in the net chemical reaction or process. “Enzyme activity” may be measured by determining the k_{cat}, K_{M} and/or ratio k_{cat}/K_{M}. “K_{M}” and “dissociation constant” interchangeably refer to the concentration of substrate that leads to half-maximal reaction rate. K_{M} describes the affinity of the substrate for the enzyme. “k_{cat}”, “catalytic rate” and “turnover number” interchangeably refer to the rate of product formation, i.e., the maximal number of product per active site per unit time. A “desired level of catalytic activity” includes catalytic rates that are the same as, lower than, and/or greater than the catalytic rates of a wild type enzyme.

“Specific binding to another molecule” is exemplified by binding of an antibody to an epitope and/or to an antigen, binding of a ligand to a receptor, binding of a transcription factor to a nucleotide sequence, etc.

“Pharmaceutical” and “physiologically tolerable” composition refers to a composition that contains pharmaceutical molecules, i.e., molecules that are capable of administration to or upon a subject and that do not substantially produce an undesirable effect such as, for example, adverse or allergic reactions, dizziness, gastric upset, toxicity and the like, when administered to a subject. Preferably also, the pharmaceutical molecule does not substantially reduce the activity of the invention's compositions. Pharmaceutical molecules include “diluent” (i.e., “carrier”) molecules such as water, saline solution, human serum albumin, oils, polyethylene glycols, aqueous dextrose, glycerin, propylene glycol or other synthetic solvents. “Carriers” may be liquid carriers (such as water, saline, culture medium, saline, aqueous dextrose, and glycols) or solid carriers (such as carbohydrates exemplified by starch, glucose, lactose, sucrose, and dextrans, anti-oxidants exemplified by ascorbic acid and glutathione, and hydrolyzed proteins).

“Plurality” means two or more.

The terms “reduce,” “inhibit,” “diminish,” “suppress,” “decrease,” and grammatical equivalents (including “lower,” “smaller,” etc.) when in reference to the level of any molecule (e.g., amino acid sequence, and nucleic acid sequence, antibody, etc.), cell, and/or phenomenon (e.g., immunogenicity, biological activity, enzyme activity, binding of two molecules, specificity of binding of two molecules, affinity of binding of two molecules, disease symptom, specificity to disease, sensitivity to disease, affinity of binding, etc.) in a first sample (or in a first subject) relative to a second sample (or relative to a second subject), mean that the quantity of molecule, cell and/or phenomenon in the first sample (or in the first subject) is lower than in the second sample (or in the second subject) by any amount that is statistically significant using any art-accepted statistical method of analysis. In one embodiment, the quantity of molecule, cell and/or phenomenon in the first sample (or in the first subject) is at least 10% lower than, at least 25% lower than, at least 50% lower than, at least 75% lower than, and/or at least 90% lower than the quantity of the same molecule, cell and/or phenomenon in the second sample (or in the second subject). In another embodiment, the quantity of molecule, cell, and/or phenomenon in the first sample (or in the first subject) is lower by any numerical percentage from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100% lower than the quantity of the same molecule, cell and/or phenomenon in the second sample (or in the second subject). In one embodiment, the first subject is exemplified by, but not limited to, a subject that has been manipulated using the invention's compositions and/or methods. In a further embodiment, the second subject is exemplified by, but not limited to, a subject that has not been manipulated using the invention's compositions and/or methods. In an alternative embodiment, the second subject is exemplified by, but not limited to, a subject to that has been manipulated, using the invention's compositions and/or methods, at a different dosage and/or for a different duration and/or via a different route of administration compared to the first subject. In one embodiment, the first and second subjects may be the same individual, such as where the effect of different regimens (e.g., of dosages, duration, route of administration, etc.) of the invention's compositions and/or methods is sought to be determined in one individual. In another embodiment, the first and second subjects may be different individuals, such as when comparing the effect of the invention's compositions and/or methods on one individual participating in a clinical trial and another individual in a hospital.

The terms “increase,” “elevate,” “raise,” and grammatical equivalents (including “higher,” “greater,” etc.) when in reference to the level of any molecule (e.g., amino acid sequence, and nucleic acid sequence, antibody, etc.), cell, and/or phenomenon (e.g., immunogenicity, biological activity, enzyme activity, binding of two molecules, specificity of binding of two molecules, affinity of binding of two molecules, disease symptom, specificity to disease, sensitivity to disease, affinity of binding, etc.) in a first sample (or in a first subject) relative to a second sample (or relative to a second subject), mean that the quantity of the molecule, cell and/or phenomenon in the first sample (or in the first subject) is higher than in the second sample (or in the second subject) by any amount that is statistically significant using any art-accepted statistical method of analysis. In one embodiment, the quantity of the molecule, cell and/or phenomenon in the first sample (or in the first subject) is at least 10% greater than, at least 25% greater than, at least 50% greater than, at least 75% greater than, and/or at least 90% greater than the quantity of the same molecule, cell and/or phenomenon in the second sample (or in the second subject). This includes, without limitation, a quantity of molecule, cell, and/or phenomenon in the first sample (or in the first subject) that is at least 10% greater than, at least 15% greater than, at least 20% greater than, at least 25% greater than, at least 30% greater than, at least 35% greater than, at least 40% greater than, at least 45% greater than, at least 50% greater than, at least 55% greater than, at least 60% greater than, at least 65% greater than, at least 70% greater than, at least 75% greater than, at least 80% greater than, at least 85% greater than, at least 90% greater than, and/or at least 95% greater than the quantity of the same molecule, cell and/or phenomenon in the second sample (or in the second subject). In one embodiment, the first subject is exemplified by, but not limited to, a subject that has been manipulated using the invention's compositions and/or methods. In a further embodiment, the second subject is exemplified by, but not limited to, a subject that has not been manipulated using the invention's compositions and/or methods. In an alternative embodiment, the second subject is exemplified by, but not limited to, a subject to that has been manipulated, using the invention's compositions and/or methods, at a different dosage and/or for a different duration and/or via a different route of administration compared to the first subject. In one embodiment, the first and second subjects may be the same individual, such as where the effect of different regimens (e.g., of dosages, duration, route of administration, etc.) of the invention's compositions and/or methods is sought to be determined in one individual. In another embodiment, the first and second subjects may be different individuals, such as when comparing the effect of the invention's compositions and/or methods on one individual participating in a clinical trial and another individual in a hospital.

The term “the same” when in reference to the level of any molecule (e.g., amino acid sequence, and nucleic acid sequence, antibody, etc.), cell, and/or phenomenon (e.g., immunogenicity, biological activity, enzyme activity, binding of two molecules, specificity of binding of two molecules, affinity of binding of two molecules, disease symptom, specificity to disease, sensitivity to disease, affinity of binding, etc.) in a first sample (or in a first subject) relative to a second sample (or relative to a second subject), means that the quantity of molecule, cell and/or phenomenon in the first sample (or in the first subject) is neither increased nor reduced relative to the quantity in the second sample (or in the second subject).

The terms “alter” and “modify” when in reference to the level of any molecule and/or phenomenon refer to an increase or decrease.

Reference herein to any numerical range expressly includes each numerical value (including fractional numbers and whole numbers) encompassed by that range. To illustrate, and without limitation, reference herein to a range of “at least 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes each whole number of 5, 6, 7, 8, 9, and 10, and each fractional number such as 5.1, 5.2, 5.3, 5.4, 5.5, 5.6, 5.7, 5.8, 5.9, etc.

### DESCRIPTION OF INVENTION

The invention provides deimmunized mutant proteins having reduced immunogenicity while exhibiting substantially the same or greater biological activity as the proteins of interest from which they are derived, as exemplified by mutant L-asparaginase that comprises amino acid substitutions compared to wild type L-asparaginase. The invention further provides methods for screening mutant enzymes (such as deimmunized enzymes) that have substantially the same or greater biological activity as a protein of interest. The invention additionally provides methods for reducing immunogenicity, without substantially reducing biological activity, of a protein of interest.

The invention's compositions and methods are useful in, for example, therapeutic applications by minimizing adverse immune responses by the host mammalian subjects to the protein of interest. Thus, the invention further provides methods for treating disease comprising administering to a subject a therapeutically effective amount of a pharmaceutical composition comprising at least one of the mutant deimmunized proteins produced by the invention's methods.

With respect to particular embodiments relating to enzyme application, a number of heterologous enzymes have been investigated for cancer treatment and other therapeutic applications, however immunogenicity issues have limited their clinical utility. Here, a new approach has been created by the inventors for enzyme (e.g., heterologous enzyme) deimmunization whereby combinatorial saturation mutagenesis is coupled with a screening strategy that capitalizes on the evolutionary biology concept of neutral drift, and combined with iterative computational prediction of T-cell epitopes to achieve extensive reengineering of a protein sequence for reduced MHC-II binding propensity without affecting catalytic and pharmacological properties.

E. coli L-asparaginase II (EcAII), the only non-human enzyme approved for repeated administration, is critical in treatment of childhood ALL, but elicits adverse antibody responses in a significant fraction of patients. The invention's neutral drift screening of combinatorial saturation mutagenesis libraries at a total of 12 positions was used to isolate an EcAII variant containing 8 amino acid substitutions within computationally predicted T-cell epitopes—of which 4 were non-conservative—while still exhibiting k_{cat}/K_{M}=10⁶M⁻¹s⁻¹ for L-Asn hydrolysis. Further, immunization of HLA transgenic mice expressing the ALL-associated DRB1*0401 allele with the engineered variant resulted in significantly reduced T-cell responses and a 10-fold reduction in anti-EcAII IgG titers relative to the existing therapeutic. This significant reduction in the immunogenicity of EcAII is clinically relevant for ALL treatment and illustrates the use of the invention's neutral drift screens to achieve large jumps in sequence space as may be required for the deimmunization of heterologous proteins.

The invention discloses methods for generating low immunogenicity variants of therapeutic proteins (including enzymes) comprising an amino acid sequence that is substantially different from that of the natural form of the protein without substantially altering the biological activity (e.g., stability of biological activity over time, enzyme catalytic activity, etc.) and/or pharmacological properties of the protein.

The invention further provides composition of matter of mutants of the therapeutic proteins as exemplified by mutants of the enzyme L-Asparaginase (Oncospar™) used to treat childhood acute lymphoblastic leukemia (ALL). Immunogenicity is a major problem in enzyme therapy for cancer and other indications. In particular in ALL up to 60% of the patients ultimately developed antibodies to L-Asparaginase that lead to reduced efficacy and discontinuation of treatment. In contrast, the invention's exemplary mutant L-Asparaginase SEQ ID NO:01 was demonstrated to elicit about 10-fold lower antibody titers in a suitable animal model representative of the human population most likely to be afflicted with ALL (using a transgenic mouse model expressing an appropriate human leukocyte antigen (HLA) allele).

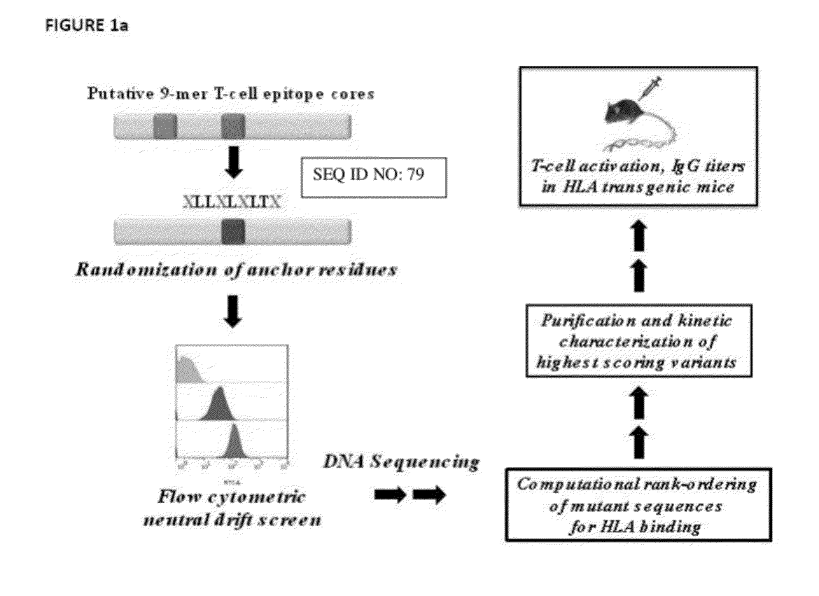

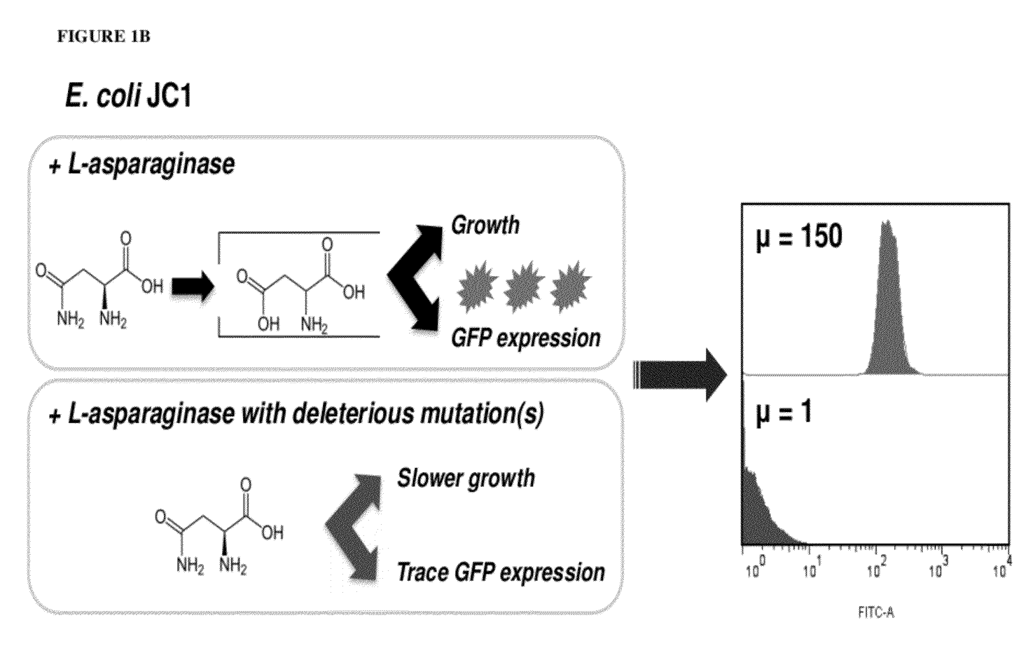

The invention takes advantage of the evolutionary biology concept of neutral drift (22-24) for the combinatorial deimmunization of an exemplary therapeutic enzyme without loss of function. Neutral drift refers to the accumulation of mutations under selective conditions that do not ultimately impact protein function. In one embodiment, for deimmunization, putative T-cell epitopes are first identified computationally (Example 1) (or experimentally), key residues important for MHC-II binding are subjected to combinatorial randomization, and the resulting libraries are subjected to the invention's neutral drift screen to isolate variants that retain wild-type (WT) function. The pools of neutral drift variants are evaluated for MHC-II binding and those that display scores indicative of reduced binding are purified and characterized biochemically. In preferred embodiments, T-cell activation assays and antibody titers in transgenic mice homozygous for disease associated HLA alleles are subsequently and/or concurrently used to evaluate T-cell epitope removal and immunogenicity, respectively (FIG. 1A). Although in this work we employed computational prediction of T-cell epitopes, the invention's neutral drift screening methodology can be coupled to the experimental detection of sequences likely to bind MHC-II, using either haplotyped human peripheral blood mononuclear cell (PBMC) pools or relevant HLA-transgenic animals.

The implementation of the strategy described above combined with high-throughput technology for the rapid isolation of mutations that have minimal or no effect on function. Since neutral drift screens for most enzymes have not been developed, the invention's methods preferably use surrogate screening methods that interrogate proteins for stability and expression rather than catalytic function (25). Manual assays, e.g. using a 96-well microtiter plate format, do not afford sufficient throughput for most purposes, while genetic selections based on complementation of auxotrophic strains to growth on selective media lacks a necessary degree of quantitation. Thus, in many instances, the expression of clones displaying significant differences in catalytic activity does not result in noticeable differences in colony formation (26). Further, the extreme adaptability of biological systems can lead to growth via mechanisms that bypass the action of the expressed heterologous protein (27-29).

The invention also provides a strategy for the rapid isolation of mutations that enhance the catalytic activity of enzymes that degrade amino acids. The inventors have developed a simple and robust screen readily applicable to a variety of proteins, including therapeutic enzymes that catalyze the depletion of amino acids or other metabolites important for disease states. FIG. 1B presents a schematic of the invention's screen as applied to the exemplary chemotherapeutic enzyme L-Asparaginase II (EcAII, EC 3.5.1.1).

Another alternative to developing low immunogenicity therapeutic enzymes is to engineer human enzymes that catalyze a therapeutically relevant reaction and exhibit appropriate pharmacological properties. In this instance, human enzymes are engineered to display the desired catalytic specificity which is not exhibited by the parental, authentic human enzyme. The engineering of novel catalytic properties is predicated by the introduction of amino acid substitutions into the parental human enzyme. Typically engineered mutant enzymes exhibiting a desired activity are isolated by screening of large combinatorial libraries. The present invention also provides methods for the identification and/or isolation of human enzyme variants that can hydrolyze target amino acids from large libraries of protein mutants expressed in microorganisms such as E. coli.

The invention is further described under A. Discussion Of Results Of Examples 1-5, B. Deimmunized Proteins, And Compositions Relating Thereto, C. Methods For Screening Mutant Deimmunized Proteins That Retain Their Biological Activity, D. Methods For Reducing Immunogenicity, and E. Therapeutic Applications.

### A. Discussion of Results of Examples 1-5

Human or humanized protein deimmunization has so far relied on the introduction of one or at most, a very limited number of conservative amino acid substitutions that attempt to remove immunogenic epitopes without substantially reducing therapeutic function. However, more drastic re-engineering of the polypeptide sequence is often required for the deimmunization of heterologous proteins (e.g., enzymes) that have not undergone tolerance induction. Introducing substantial changes in the primary sequence of enzymes without affecting stability of function poses a significant challenge.

Data herein shows that the use of the invention's combinatorial mutagenesis and screens that directly interrogate protein function can be exploited to take large leaps in sequence space and thus generate variant polypeptides with reduced propensity to bind to MHC-II and elicit T-dependent antibody responses. The exemplary EcAII 3.1.E2 mutant contained 8 amino acid substitutions, 3 of which are not observed in any of the nearly 500 bacterial type II asparaginases in the database, yet retained near WT catalytic efficiency and stability. EcAII 3.1.E2 exhibited substantially reduced immunogenicity in HLA-transgenic mice and thus constitutes a very promising candidate for alleviating adverse responses in the treatment of childhood ALL. Further, the development of an asparaginase displaying reduced immunogenicity is especially beneficial for longer term treatment in adult ALL and/or for relapsing patients.

The invention's screening strategy provided herein by the inventors may be readily applied for the combinatorial deimmunization of any protein, including heterologous therapeutic enzymes such as enzymes used in cancer treatment that function by systemic amino acid depletion, including L-asparaginase, Arginine deiminase, L-methioninase, phenylalanine ammonia lyase (PAL), urate oxidase, ecotin, glutamine deaminase, monoclonal antibodies and/or fragments thereof.

Likewise, the invention's screens may be readily used for the engineering of human enzymes capable of degrading a particular amino acid of interest with a desired catalytic rate. In on embodiment, the effect of mutations on protein expression may be accounted for by constructing a fusion of the target protein to a fluorescent protein that emits at a different wavelength (e.g. red fluorescent protein; RFP, which incidentally can be secreted in the bacterial periplasm in a fluorescent form (51)). The use of fluorescent protein fusions to monitor expression and folding in vivo is well established (52), and thus two-color sorting could be used to select for both expression and activity simultaneously. Ultimately however, the stability of the isolated mutants is preferably additionally determined in medium throughput assays (e.g. 96 well plates). Finally, while in this work by the inventors employed a computational approach for monitoring immunogenicity, experimental methods for identifying T-cell epitopes can also be applied to monitor the immunogenicity propensity of mutant proteins.

### B. Deimmunized Proteins, and Compositions Relating Thereto

The invention's screening strategy together with iterative combinatorial saturation mutagenesis was used herein to isolate an exemplary mutant, named 3.1.E2 containing a total of 8 amino acid substitutions, yet retaining a k_{cat} substantially identical to the parent wild-type enzyme with just a 3-fold increase in K_{M} (k_{cat}=24 s⁻¹, K_{M}=23 μM, k_{cat}/K_{M}=1.0×10⁶ M⁻¹s⁻¹). 3.1.E2 further was stable in serum for over 10 days and could be expressed at a high yield (>30 mg/L shake flask culture.

In one embodiment, the invention provides a mutant L-asparaginase that A) comprises 8 amino acid substitutions that correspond to substitution of wild type L-asparaginase SEQ ID NO:03 (that is encoded by SEQ ID NO:04) 1) methionine at position 115 with valine (M115V), 2) Serine at position 118 with proline (S118P), 3) Serine at position 120 with arginine (S120R), 4) alanine at position 123 with proline (A123P), 5) isoleucine at position 215 with valine (1215V), 6) asparagine at position 219 with glycine (N219G), 7) glutamine at position 307 with threonine (Q307T), and 8) glutamine at position 312 with asparagine (Q312N), B) has substantially the same or greater enzyme activity as wild type L-asparaginase SEQ ID NO:03, and C) has reduced immunogenicity compared to wild type L-asparaginase SEQ ID NO:03.

Data herein show that the exemplary mutant L-asparaginase 3.1.E2 (SEQ ID NO:01) has 10-fold reduced immunogenicity as measured by anti-EcAII (i.e., anti-wild type L-asparaginase) IgG titers compared to the wild-type L-Asparaginase (SEQ ID NO:03) (Example 5), while retaining substantially the same enzyme activity (as measured by k_{cat} for the enzyme substrate L-asparagine), and stability in serum for over 10 days (Example 4)

In particular, data herein show that the immunogenicity of purified, low endotoxin preparations of wild-type EcAII and 3.1.E2 were evaluated in transgenic mice expressing human HLA-DRB1*0401 under the mouse MHC-II promoter and deficient in the endogenous murine MHC-II locus. As a stringent test of the potential for immunogenicity, mice were immunized with a strong adjuvant (Complete Freund's Adjuvant) to induce robust CD4+ T-cell responses. The HLA transgenic mice were immunized with either wild-type EcAII or 3.1.E2 and T-cell responses were measured in draining lymph node cells by cytokine ELISPOT assays for IFN-γ levels following recall with either overlapping 20-mer synthetic peptides corresponding to wild type 3.1.E2 sequences, or with the enzyme used for initial immunization. Deimmunization resulted in a significant decrease in T-cell activation Importantly, mice immunized with 3.1.E2 displayed a statistically significant (p=0.02) 10-fold reduction in anti-EcAII IgG titer relative to mice receiving the wild-type enzyme.

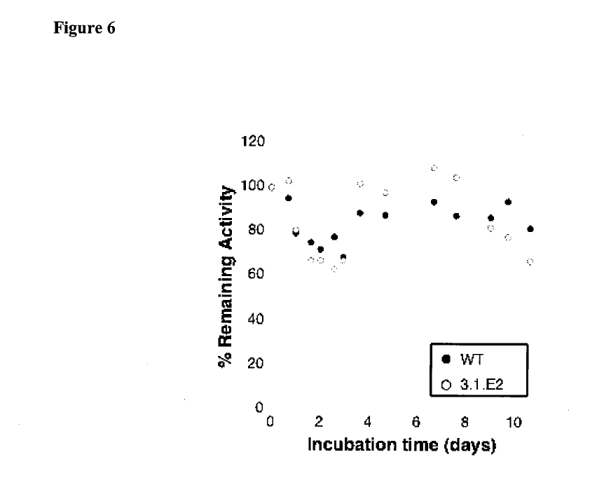

While not intending to limit the activity of the mutant enzyme, in one embodiment, the mutant L-asparaginase has substantially the same or greater stability of enzyme activity in serum as wild type L-asparaginase SEQ ID NO:03. For example, data herein show that the exemplary mutant L-asparaginase SEQ ID NO:01 (3.1.E2) has substantially the same stability of enzyme activity in serum over approximately 10 days when compared to wild type L-asparaginase SEQ ID NO:03 (FIG. 6).

In preferred embodiments, the mutant L-asparaginase has k_{cat}/K_{M} equal to or greater than 10⁴ M⁻¹ s⁻¹. Data herein show that the exemplary EcAII-G57A mutant L-asparaginase SEQ ID NO:01 has k_{cat}/K_{M} (L-aspartic acid β-hydroxomate (AHA)) of 2.2×10⁵ M⁻¹s⁻¹ (Example 3). L-asparaginase mutant enzymes with k_{cat}/K_{M} (L-Asn)>10⁶ M⁻¹ s⁻¹ should be more than sufficient for therapeutic purposes given that circulating L-Asn is depleted to negligible levels within minutes following the administration of a therapeutic dose of WT EcAII (43), and remain low for weeks afterwards (32, 43). Therefore, even though enzymes with k_{cat}/K_{M} (L-Asn) up to 3 to 4-fold below that of the WT enzyme might result in marginally slower initial depletion of serum L-Asn, they should not affect the longer term maintenance of low serum L-Asn levels which is the therapeutically relevant parameter.

In particular embodiments, the mutant L-asparaginase comprises SEQ ID NO:01 (3.1.E2). In some embodiments, the mutant L-asparaginase is recombinant. In alternative embodiments, the mutant L-asparaginase is purified.

The invention also provides pharmaceutical compositions comprising any one or more of the mutant L-asparaginase enzymes described herein and a carrier.

The invention further provides recombinant nucleotide sequences encoding any one or more of the mutant L-asparaginase enzymes described herein. In one embodiment, the nucleotide sequence comprises SEQ ID NO:02. In a further embodiment, the nucleotide sequence is operably linked to a promoter. In a preferred embodiment, the nucleotide sequence is comprised in an expression vector. In an alternative embodiment, the nucleotide sequence is purified.

The invention also provides expression vectors that comprise one or more nucleotide sequences encoding any one or more of the mutant L-asparaginase enzymes described herein.

The invention further includes transgenic cells comprising expression vectors encoding any one or more of the mutant L-asparaginase enzymes described herein. The invention's vectors (i.e., plasmids, linear DNA, encapsidated virus, etc.) may be introduced into cells using techniques well known in the art. The term “introducing” a nucleic acid sequence into a cell refers to the introduction of the nucleic acid sequence into a target cell to produce a “transformed” or “transgenic” cell. Methods of introducing nucleic acid sequences into cells are well known in the art. For example, where the nucleic acid sequence is a plasmid or naked piece of linear DNA, the sequence may be “transfected” into the cell using, for example, calcium phosphate-DNA co-precipitation, DEAE-dextran-mediated transfection, polybrene-mediated transfection, electroporation, microinjection, liposome fusion, lipofection, protoplast fusion, and biolistics. Alternatively, where the nucleic acid sequence is encapsidated into a viral particle, the sequence may be introduced into a cell by “infecting” the cell with the virus.

Transformation of a cell may be stable or transient. The terms “transient transformation” and “transiently transformed” refer to the introduction of one or more nucleotide sequences of interest into a cell in the absence of integration of the nucleotide sequence of interest into the host cell's genome. Transient transformation may be detected by, for example, enzyme-linked immunosorbent assay (ELISA) that detects the presence of a polypeptide encoded by one or more of the nucleotide sequences of interest. Alternatively, transient transformation may be detected by detecting the activity of the protein encoded by the nucleotide sequence of interest. The term “transient transformant” refer to a cell that has transiently incorporated one or more nucleotide sequences of interest.

In contrast, the terms “stable transformation” and “stably transformed” refer to the introduction and integration of one or more nucleotide sequence of interest into the genome of a cell. Thus, a “stable transformant” is distinguished from a transient transformant in that, whereas genomic DNA from the stable transformant contains one or more heterologous nucleotide sequences of interest, genomic DNA from the transient transformant does not contain the heterologous nucleotide sequence of interest. Stable transformation of a cell may be detected by Southern blot hybridization of genomic DNA of the cell with nucleic acid sequences that are capable of binding to one or more of the nucleotide sequences of interest. Alternatively, stable transformation of a cell may also be detected by the polymerase chain reaction of genomic DNA of the cell to amplify the nucleotide sequence of interest.

The invention's cells are exemplified by transgenic cells for screening enzyme mutants, such as E. coli JC1 (MC1061 ΔaspCΔtyrBΔansAΔansBΔiaaA) in which the genes required for L-Asp biosynthesis (aspC, tyrB) and the three genes required for endogenous L-asparaginase enzymes were deleted. This transgenic cell was used in one embodiment of the invention's neutral drift screen for EcAII.

The invention's cells are also exemplified by transgenic cells for use as host cells for expression vectors to produce the invention's therapeutic mutant enzymes, including without limitation, avian cells, insect cells and mammalian cells. In one embodiment, the cells are in vitro. In one embodiment, the cells are non-human and are exemplified, but not limited to, rabbit primary neuronal cells, Madin Darby bovine kidney (MDBK) cells ATCC# ccl-22; Swine kidney cells (SK6-M, described in European Patent 0 351 901 B1); LM cells (mouse fibroblast), ATCC# ccl-1.2; NCTC 3526 cells (rhesus monkey kidney), ATCC# ccl-7.2; BHK-21 cells (golden hamster kidney), ATCC# ccl-10; PK 15 cells (pig kidney), ATCC# ccl-33; MDCK cells (dog kidney), ATCC# ccl-34; PtK1 cells (kangaroo rat kidney), ATCC# ccl-35; Rk 13 cells (rabbit kidney), ATCC# ccl-37; Dede cells (Chinese hamster lung fibroblast), ATCC# ccl-39; Bu (IMR31) cells (bison lung fibroblast), ATCC# ccl-40; FHM cells (minnow epithelial), ATCC# ccl-42; LC-540 cells (rat Leydig cell tumor), ATCC# ccl-43; TH-1 cells (turtle heart epithelial), ATCC# ccl-50; E. Derm (NBL-6) cells (horse fibroblast), ATCC# ccl-57; MvLn cells (mink epithelial), ATCC# ccl-64; Ch1 Es cells (goat fibroblast), ATCC# ccl-73; Pl I Nt cells (raccoon fibroblast), ATCC# ccl-74; Sp I k cells (dolphin epithelial), ATCC# ccl-78; CRFK cells (cat epithelial), ATCC# ccl-94; Gekko Lung 1 cells (lizard-gekko epithelial), ATCC# ccl-111; Aedes Aegypti cells (mosquito epithelial), ATCC# ccl-125; ICR 134 cells (frog epithelial), ATCC# ccl-128; Duck embryo cells (duck fibroblast), ATCC# ccl-141; and DBS Fcl-1 cells (monkey lung fibroblast), ATCC# ccl-161.

In another embodiment, the cells are human and are exemplified, but not limited to, U937 cells (macrophage), ATCC# crl 1593.2; A-375 cells (melanoma/melanocyte), ATCC# crl-1619; KLE cells (uterine endometrium), ATCC# crl-1622; T98G cells (glioblastoma), ATCC# crl-1690; CCF-STTG1 cells (astrocytoma), ATCC# crl-1718; HUV-EC-C cells (vascular endothelium), ATCC# CRL-1730; UM-UC-3 cells (bladder), ATCC# crl-1749; CCD841-CoN cells (colon, ATCC# crl-1790; SNU-423 cells (hepatocellular carcinoma), ATCC# crl-2238; WI38 cells (lung, normal), ATCC# crl-75; Raji cells (lymphoblastoid), ATCC# ccl-86; BeWo cells (placenta, choriocarcinoma), ATCC# ccl-98; HT1080 cells (fibrosarcoma), ATCC# ccl-121; MIA PaCa2 cells (pancreas), ATCC# crl-1420; CCD-25SK cells (skin fibroblast), ATCC# crl-1474; ZR75-30 cells (mammary gland), ATCC# crl-1504; HOS cells (bone osteosarcoma), ATCC# crl-1543; 293-SF cells (kidney), ATCC# crl-1573; LL47 (MaDo) cells (normal lymphoblast), ATCC# ccl-135; and HeLa cells (cervical carcinoma), ATCC# ccl-2.

### C. Methods For Screening Mutant Deimmunized Proteins That Retain Their Biological Activity

The invention provides a method for identifying a mutant deimmunized protein that has substantially the same or greater biological activity as a protein of interest, comprising A) providing i) a first plurality of first expression vectors, wherein each expression vector comprises in operable combination 1) a first nucleotide sequence encoding a mutant of a protein of interest, wherein the protein of interest comprises one or more epitope sequence, and wherein the mutant protein contains one or more mutations in one or more of the epitope sequence, 2) a reporter nucleotide sequence, and 3) a promoter, ii) a transgenic cell that lacks expression of a biologically active the protein of interest, and B) transfecting the transgenic cell with the first plurality of first expression vectors to produce a first plurality of populations of transfected transgenic cells, wherein each population of the first plurality of populations of transfected transgenic cells comprises one of the first expression vectors, C) culturing the first plurality of populations of transfected transgenic cells under conditions for expression of the first nucleotide sequence and of the reporter nucleotide sequence, D) detecting expression of the reporter nucleotide sequence in the first plurality of populations of transfected transgenic cells, wherein the level of the expression of the reporter nucleotide sequence correlates with the level of biological activity of the mutant protein that is encoded by the operably linked first nucleotide sequence, and E) determining the immunogenicity in the first plurality of populations of transfected transgenic cells of the expressed mutant protein, wherein i) detecting substantially the same or greater biological activity of the mutant protein compared to the protein of interest, and ii) detecting reduced immunogenicity of the mutant protein compared to the protein of interest, identifies the mutant protein as a deimmunized protein that has substantially the same or greater biological activity as the protein of interest (FIG. 1).

Thus, in one embodiment of the invention's neutral drift screen for the exemplary EcAII, the inventors first constructed E. coli JC1 (MC1061 ΔaspCΔtyrBΔansAΔansBΔiaaA) in which the genes required for L-Asp biosynthesis (asp C, tyrB) and the three genes required for endogenous L-asparaginase enzymes were deleted. In this manner, the normal protein biosynthesis and viability of E. coli JC1 was made dependent upon recombinant expression of an active EcAII. To enable an additional level of quantitation, E. coli JC1 cells were transformed with a plasmid expressing GFP under an IPTG inducible promoter, grown in media containing all 20 amino acids to late exponential phase, washed and transferred for a short time period to media with 19aa (no L-Asp) and IPTG to induce GFP synthesis. Following the addition of IPTG, GFP synthesis—and hence cell fluorescence—was dependent on the availability of L-Asp which in turn was proportional to EcAII enzymatic activity. Utilizing this assay, the inventors found that intracellular GFP fluorescence correlated well with the activity of a panel of recombinantly expressed EcAII variants displaying up to two orders of magnitude differences in catalytic efficiency.

In preferred embodiments, the invention's screening strategy is combined with iterative combinatorial saturation mutagenesis by, for example, sequentially mutating more amino acids in the identified deimmunized mutant. In particularly preferred embodiments, the deimmunized proteins that are identified by the invention's randomization and neutral drift screening methods are subjected to further randomization and neutral drift screening in which additional putative epitopes are mutated and then screened for biological activity and immunogenicity compared to the protein of interest. This is illustrated in Example 3 and Example 4 with respect to L-asparaginase, in which the T-cell epitope regions M₁₁₅RPSTSMSA, I₂₁₆VYNYANAS, and V₃₀₄LLQLALTQ (designated M₁₁₅, I₂₁₆, and V₃₀₄ where these three residues correspond to the respective P1 positions) were sequentially subjected to randomization and neutral drift screening, starting with M₁₁₅ and continuing with I₂₁₆ and finally V₃₀₄. Some advantages of this sequential approach is that it: (i) ensures complete library coverage for each individual epitope; (ii) evaluates the relative plasticity of different regions of the protein to amino acid mutations (e.g., substitutions), and (iii) simplifies the structural interpretation of any observed changes in the activity of isolated mutants.

Thus, in one embodiment, the invention's methods further comprise F) providing i) a second plurality of second expression vectors, wherein each expression vector of the second plurality of expression vectors comprises in operable combination, 1) a second nucleotide sequence encoding a variant of the identified mutant protein, wherein the variant protein contains additional one or more mutations in the one or more epitope sequence of the identified mutant protein, 2) a reporter nucleotide sequence, and 3) a promoter, ii) a transgenic cell that lacks expression of a biologically active the protein of interest, and G) transfecting the transgenic cell with the second plurality of expression vectors to produce a second plurality of populations of transfected transgenic cells, wherein each population of the second plurality of populations of transfected transgenic cells comprises one of the second expression vectors, H) culturing the second plurality of populations of transfected transgenic cells under conditions for expression of the second nucleotide sequence and the reporter nucleotide sequence, I) detecting expression of the reporter nucleotide sequence in the second plurality of populations of transfected transgenic cells, wherein the level of the expression of the reporter nucleotide sequence correlates with the level of biological activity of the variant protein that is encoded by the operably linked second nucleotide sequence, and J) determining the immunogenicity in the plurality of populations of transfected transgenic cells of the expressed variant protein, wherein i) detecting substantially the same or greater biological activity of the variant protein compared to the protein of interest, and ii) detecting reduced immunogenicity of the variant protein compared to the protein of interest, identifies the variant protein as a deimmunized protein that has substantially the same or greater biological activity as the protein of interest.

In preferred embodiments, the invention's screening strategy in combination with iterative combinatorial saturation mutagenesis further comprises detecting the stability of the biological activity of the mutant protein. In particularly preferred embodiments, this involves detecting substantially the same or greater stability of the biological activity of the mutant protein compared to the stability of the protein of interest.

In particularly preferred embodiments, the invention's screening strategy in combination with iterative combinatorial saturation mutagenesis further comprises purifying the identified mutant deimmunized protein.

In an alternative embodiment, the invention's screening strategy in combination with iterative combinatorial saturation mutagenesis further comprises detecting one or more mutation in the epitope sequence of the purified mutant deimmunized protein. This may be done to evaluate epitope removal using T-cell activation assays (FIG. 1A).

In further embodiments, the invention's screening strategy in combination with iterative combinatorial saturation mutagenesis further comprises determining the immunogenicity of the purified mutant deimmunized protein. This may be done to confirm the deimmunization of the purified mutant deimmunized protein, and may be achieved using methods known in the art, such as by determining antibody titers in animal models (FIG. 1A).

While the invention's methods are illustrated by L-asparaginase enzyme mutants, they are nonetheless applicable to deimmunization of any protein of interest while maintaining substantially the same or greater biological activity of the protein of interest. In some embodiments, the protein of interest is an enzyme, and the transgenic cell further lacks expression of a product produced by the enzyme activity of a wild type of the enzyme of interest. This may be accomplished by using transgenic cells containing a mutation (e.g., gene deletion) in endogenous genes encoding enzymes for the biosynthesis of the enzyme's product. This is illustrated by deletion in mutant E. coli JC1 of the 2 genes (asp C, tyrB) that are required for the biosynthesis of L-Asp, which is the product of endogenous L-asparaginase enzyme activity (Example 3).

While not intending to limit the type of mutation in the epitope sequence of protein of interest, in some embodiments the mutation in the epitope sequence comprises one or more amino acid substitutions.

In some embodiments, the epitope sequence includes one or more T-cell epitopes sequence, and/or one or more B-cell epitope sequence.

In some preferred embodiments, the epitope sequence is a T-cell epitope sequence. “T-cell epitope” is an epitope that specifically binds to the major histocompatibility complex (MHC)-II and elicits a T cell-dependent (Td) immune response. Putative T-cell epitopes are identified computationally using methods known in the art, and exemplified herein such as the Immune Epitope Database (IEDB) consensus method (Example 3). In an alternative embodiment, putative T-cell epitopes may be identified by experimental detection of sequences likely to bind MHC-II, using either haplotyped peripheral blood mononuclear cell (PMBC) pools or relevant HLA-transgenic animals.

Thus, the invention's methods may further include determining the immunogenicity of the mutant protein by detecting binding of the T-cell epitope sequence to the to the major histocompatibility complex (MHC)-II. Methods for detecting MHC-II binding of T-cell epitopes are known in the art and exemplified herein, such as in Example 5.

In some embodiments, the mutant protein that contains one or more mutations in the epitope sequence is produced by randomization of one or more nucleotides in the first nucleotide sequence encoding the mutant protein (Example 4).

The invention utilizes reporter nucleotide sequences in the expression vectors. “Reporter sequence” and “marker sequence” are used interchangeably to refer to a DNA, RNA, and/or polypeptide sequence that is detectable in any detection system, including, but not limited to enzyme (e.g., ELISA, as well as enzyme-based histochemical assays), fluorescent, radioactive, and luminescent systems. Exemplary reporter genes include, for example, β-glucuronidase gene, green fluorescent protein (GFP) gene, E. coli β-galactosidase (LacZ) gene, Halobacterium β-galactosidase gene, E. coli luciferase gene, Neuropsora tyrosinase gene, Aequorin (jellyfish bioluminescence) gene, human placental alkaline phosphatase gene, and chloramphenicol acetyltransferase (CAT) gene. Reporter genes are commercially available, such as from Clontech, Invitrogen, and Promega. It is not intended that the present invention be limited to any particular detection system or label. In particular embodiments, the reporter nucleotide sequence comprises a gene encoding a fluorescent protein, exemplified but not limited to, green fluorescent protein (Example 2) and red fluorescent protein (Chen et al (2005) Mol. Microbiol. 55(4):1085-1103).

The invention's methods are contemplated to not adversely impact the biological activity of the proteins of interest. In some embodiments, biological activity of the protein of interest is exemplified by enzyme activity and specific binding to a second molecule.

The invention's methods are illustrated using the exemplary mutant L-Asparaginase SEQ ID NO:01. However, they are equally application to any protein of interest, such as an enzyme of interest and binding protein of interest.

In one embodiment, the protein of interest is an enzyme, such as an amino acid degrading enzyme exemplified by L-asparaginase, arginine deiminase, L-methioninase, phenylalanine ammonia lyase (PAL), urate oxidase, ecotin, glutamine deaminase and/or enzyme active fragments thereof, as well as antibodies and/or antigen-binding fragments thereof.

“Asparaginase,” “L-asparaginase,” and “EcAII” interchangeably refer to an enzyme that degrades L-asparagine (L-Asn), and is exemplified by the E. coli wild-type amino acid sequence SEQ ID NO:03. EcAII has been a cornerstone component of chemotherapeutic protocols for the treatment of ALL for over 40 years (30-33). In ALL, lymphoblasts lack or express low levels of L-asparagine synthetase (AS) (34) and therefore require the uptake of L-Asn from serum for cell proliferation (6). EcAII catalyzes the hydrolysis of L-Asn to L-Asp and ammonia with k_{cat}/K_{M}=3.3×10⁶ M⁻¹s⁻¹ (as calculated herein) resulting in the systemic depletion of serum L-Asn (7, 35, 36), which in turn induces apoptosis of ALL lymphoblasts (37, 38). However, antibody responses to EcAII have been reported in up to 60% of patients (39). The prior art's primary strategy for managing the disease in patients with adverse immune responses to EcAII is treatment with the Erwinia caratovora L-Asparaginase II, which although is non-cross-reactive with anti-EcAII antibodies (40), is also highly immunogenic and clinically inferior to EcAII with respect to both event-free survival and overall survival rates at 6 years (41).

The invention's neutral drift screening strategy together with iterative combinatorial saturation mutagenesis to isolate a mutant, named 3.1.E2 (SEQ ID NO:01) containing a total of 8 amino acid substitutions, yet retaining a k_{cat} identical to the parent enzyme with just a 3-fold increase in K_{M} (k_{cat}=24 s⁻¹, K_{M}=23 μM, k_{cat}/K_{M}=1.0×10⁶ M⁻¹s⁻¹). 3.1.E2 further was stable in serum for over 10 days and could be expressed at a high yield (>30 mg/L shake flask culture).

The deimmunized mutant of L-Asparaginase (exemplified by 3.1.E2 listed as SEQ ID NO:01) may be used for the prevention and/or treatment of “acute lymphoblastic leukemia” (“ALL”).

“Arginine deiminase” (e.g., from Mycoplasma arginini), which catalyzes breakdown of L-Arginine into citrulline and ammonia, which is exemplified by the wild-type amino acid sequence UniProt #P23793 (SEQ ID NO:05), the deimmunized mutant of which may be used for the prevention and/or treatment of hepatocellular carcinomas.

“L-methioninase” (e.g., from Pseudomonas putida), which catalyzes breakdown of L-Methionine to methanethiol, 2-oxobutanoate, and ammonia, which is exemplified by the wild-type amino acid sequence UniProt #P13254 (SEQ ID NO:06), the deimmunized mutant of which may be used for the prevention and/or treatment of central nervous system (CNS) cancers (See, for example, U.S. Pat. Nos. 6,312,939, 6,737,259 and 5,690,929).

“Phenylalanine ammonia lyase” (“PAL”) (e.g., from Anabeaena variabilis), which catalyzes breakdown of L-Phenylalanine to cinnamate and ammonia, which is exemplified by the wild-type amino acid sequence UniProt #Q3M5Z3 (SEQ ID NO:07), the deimmunized mutant of which may be used for enzyme substitution prevention and/or treatment of phenylketonuria (PKU) and/or of cancer, based on its ability to limit the nutrient supply of phenylalanine to cancer cells and thereby inhibit neoplastic growth (Kakkis et al., U.S. Pat. No. 7,790,433).

“Urate oxidase” (e.g., from Aspergillus flavus), which catalyzes conversion of uric acid to allantoin, which is exemplified by the wild-type amino acid sequence UniProt #Q00511 (SEQ ID NO:08), the deimmunized mutant of which may be used for the prevention and/or treatment of tumor lysis syndrome.

“Ecotin” (e.g., from E. coli), which catalyzes inhibition of pancreatic serine proteases, which is exemplified by the wild-type amino acid sequence UniProt #P23827 (SEQ ID NO:09), the deimmunized mutant of which may be used for the prevention and/or treatment of cancer and/or other pathological states for which S1A proteases have been implicated (examples of S1A proteases are cited in Stoop & Craik, Nature Biotechnology, 2003, V. 21, p. 1063-8).

“Glutamine deaminase” (e.g., from Burkholderia xenovorans), which degrades glutamine, is exemplified by the wild-type amino acid sequence UniProt #Q145Z3 (SEQ ID NO:10).

In another embodiment, the protein of interest is a binding protein of interest, such as antibody and/or antigen-binding fragment thereof, ligand, and transcription factor.

In particular embodiments, the binding protein of interest comprises an antibody and/or antigen-binding fragment thereof. “Antibody” refers to an immunoglobulin (e.g., IgG, IgM, IgA, IgE, IgD, etc.) and/or portion thereof that contains a “variable domain” (also referred to as the “F_{V} region”) for binding to antigens. More specifically, variable loops, three each on the light (V_{L}) and heavy (V_{H}) chains are responsible for binding to the antigen. These loops are referred to as the “complementarity determining regions” (“CDRs”) and “idiotypes.” In one embodiment, the invention's antibodies are monoclonal antibodies produced by hybridoma cells.

In particular, the invention contemplates antibody fragments that contain the idiotype (“antigen-binding fragment”) of the antibody molecule. For example, such fragments include, but are not limited to, the Fab region, F(ab′)2 fragment, pFc′ fragment, and Fab′ fragments.

The “Fab region” and “fragment, antigen binding region,” interchangeably refer to portion of the antibody arms of the immunoglobulin “Y” that function in binding antigen. The Fab region is composed of one constant and one variable domain from each heavy and light chain of the antibody. Methods are known in the art for the construction of Fab expression libraries (Huse et al., Science, 246:1275-1281 (1989)) to allow rapid and easy identification of monoclonal Fab fragments with the desired specificity. In another embodiment, Fc and Fab fragments can be generated by using the enzyme papain to cleave an immunoglobulin monomer into two Fab fragments and an Fc fragment. The enzyme pepsin cleaves below the hinge region, so a “F(ab′)2 fragment” and a “pFc′ fragment” is formed. The F(ab′)2 fragment can be split into two “Fab′ fragments” by mild reduction.

The “Fc” and “Fragment, crystallizable” region interchangeably refer to portion of the base of the immunoglobulin “Y” that function in role in modulating immune cell activity. The Fc region is composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. By doing this, it mediates different physiological effects including opsonization, cell lysis, and degranulation of mast cells, basophils and eosinophils. In an experimental setting, Fc and Fab fragments can be generated in the laboratory by cleaving an immunoglobulin monomer with the enzyme papain into two Fab fragments and an Fc fragment.

Also contemplated are chimeric antibodies. As used herein, the term “chimeric antibody” contains portions of two different antibodies, typically of two different species. See, e.g.: U.S. Pat. No. 4,816,567 to Cabilly et al.; U.S. Pat. No. 4,978,745 to Shoemaker et al.; U.S. Pat. No. 4,975,369 to Beavers et al.; and U.S. Pat. No. 4,816,397 to Boss et al.

The invention also contemplates “humanized antibodies,” i.e., chimeric antibodies that have constant regions derived substantially or exclusively from human antibody constant regions, and variable regions derived substantially or exclusively from the sequence of the variable region from a mammal other than a human. Humanized antibodies preferably have constant regions and variable regions other than the complement determining regions (CDRs) derived substantially or exclusively from the corresponding human antibody regions and CDRs derived substantially or exclusively from a mammal other than a human. Humanized antibodies may be generated using methods known in the art, including using human hybridomas (Cote et al., Proc. Natl. Acad. Sci. U.S.A. 80:2026-2030 (1983)) or by transforming human B cells with EBV virus in vitro (Cole et al., in Monoclonal Antibodies and Cancer Therapy, Alan R. Liss, pp. 77-96 (1985)). Additional methods include, for example, generation of transgenic non-human animals which contain human immunoglobulin chain genes and which are capable of expressing these genes to produce a repertoire of antibodies of various isotypes encoded by the human immunoglobulin genes (U.S. Pat. Nos. 5,545,806; 5,569,825 and 5,625,126). Humanized antibodies may also be made by substituting the complementarity determining regions of, for example, a mouse antibody, into a human framework domain (PCT Pub. No. WO92/22653). Chimeric antibodies containing amino acid sequences that are fused to constant regions from human antibodies, or to toxins or to molecules with cytotoxic effect, are known in the art (e.g., U.S. Pat. Nos. 7,585,952; 7,227,002; 7,632,925; 7,501,123; 7,202,346; 6,333,410; 5,475,092; 5,585,499; 5,846,545; 7,202,346; 6,340,701; 6,372,738; 7,202,346; 5,846,545; 5,585,499; 5,475,092; 7,202,346; 7,662,387; 6,429,295; 7,666,425; and 5,057,313).

Antibodies that are specific for a particular antigen may be screened using methods known in the art, for example, by radioimmunoassay, ELISA (enzyme-linked immunosorbent assay), “sandwich” immunoassays, immunoradiometric assays, gel diffusion precipitin reactions, immunodiffusion assays, in situ immunoassays (e.g., using colloidal gold, enzyme or radioisotope labels), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In alternative embodiments, the binding protein of interest comprises a ligand. “Ligand” refers to a molecule that binds to a second molecule such as to a receptor, antibody, etc. Thus, the term “ligand of a cell receptor” refers to a molecule that binds to a cell receptor. In one embodiment, the ligand of a cell receptor comprises a growth factor, such as one or more of epidermal growth factor, insulin-like growth factor, fibroblast growth factor, and vascular endothelial growth factor. In a particularly preferred embodiment, the growth factor comprises basic fibroblast growth factor (bFGF) (GenBank No. AAA52533) encoded by CDS 467-934 of the nucleic acid sequence (GenBank No. J04513.1).

In particular embodiments, the binding protein of interest comprises a sequence-specific DNA-binding factor. “Transcription factor” and “sequence-specific DNA-binding factor” interchangeably refer to a protein that binds to specific DNA sequences, thereby controlling the movement (or transcription) of genetic information from DNA to mRNA by promoting (as an activator), or blocking (as a repressor) the recruitment of RNA polymerase to specific genes. Transcription factors are characterized by containing one or more DNA-binding domains (DBDs), which attach to specific sequences of DNA adjacent to the genes that they regulate.

Transcription factors that are associated with disease include, for example, the STAT family transcription factors associated with breast cancer, the HOX family transcription factors associated with a variety of cancers, the tumor suppressor factor p53 associated with Li-Fraumeni syndrome, the MECP2 transcription factor associated with Rett syndrome, a neurodevelopmental disorder, hepatocyte nuclear factors (HNFs) and insulin promoter factor-1 (IPF1/Pdx1) that are associated with a rare form of diabetes called MODY (Maturity onset diabetes of the young), the FOXP3 transcription factor associate with a rare form of autoimmune disease called IPEX, and the FOXP2 transcription factor that is associated with developmental verbal dyspraxia, a disease in which individuals are unable to produce the finely coordinated movements required for speech.

The mutant proteins screened by the invention's methods have reduced immunogenicity compared to the protein of interest from which they were derived. In one embodiment, reduced immunogenicity comprises reduced T-cell activation. In another embodiment, reduced immunogenicity comprises from 1 to 10,000 fold lower (including from 1 to 1,000, from 1 to 100, from 1 to 10, and from 1 to 5 lower) immunogenicity of the mutant protein compared to immunogenicity of the protein of interest. For example, data herein demonstrate that the exemplary mutant L-asparaginase 3.1.E2 (SEQ ID NO:01) a 10-fold reduced immunogenicity compared to the wild-type L-Asparaginase (SEQ ID NO:03) (Example 2).

The invention provides pharmaceutical compositions comprising one or more mutant protein identified by the methods described herein, and a carrier.

Also provided are recombinant nucleotide sequences encoding one or more mutant protein identified by the methods described herein. In some embodiments, the nucleotide sequence is operably linked to a promoter. In alternative embodiments, the nucleotide sequence is comprised in an expression vector. In yet further embodiments, the nucleotide sequence is purified.

The invention also provides expression vectors that comprise a nucleotide sequence encoding one or more mutant protein identified by the methods disclosed herein.

Also contemplated by the invention are transgenic cells comprising one or more expression vectors for expression of one or more mutant protein identified by the methods described herein.

### D. Methods For Reducing Immunogenicity

The invention provides a method for reducing immunogenicity of a protein of interest without substantially] reducing biological activity of the protein of interest, comprising a) identifying a mutant of the protein of interest using any of the methods described herein, b) determining the amino acid sequence of one or more the epitope sequence in the identified mutant protein, and c) producing a variant protein of interest (or portion thereof) that contains the determined epitope sequence. The variant protein of interest may be produced by chemical synthesis, expression in a cell using recombinant expression vectors, etc.

The invention contemplates a pharmaceutical composition comprising one or more variant protein of interest produced by the methods described herein, and a carrier.

The invention also provides recombinant nucleotide sequences encoding one or more variant protein of interest produced by the methods of the invention. In some embodiments, the nucleotide sequence is operably linked to a promoter and/or comprised in an expression vector and/or is purified.

The invention further provides expression vectors that comprise one or more nucleotide sequence encoding one or more variant protein of interest produced by the invention's methods.

Also provided by the invention are transgenic cells comprising one or more of the invention's expression vectors.

E. Therapeutic Applications

The invention's compositions may be used for therapeutic application, such as in a method for treating disease comprising administering to a mammalian subject in need thereof a therapeutically effective amount of a pharmaceutical composition comprising at least one protein selected from one or more of a) the mutant L-asparaginase described herein, b) the mutant deimmunized protein identified by the invention's methods, and c) the variant protein of interest produced by the invention's methods. One advantage is that the invention's mutant proteins retain the same or higher activity while possessing reduced immunogenicity, thereby minimizing adverse immune responses by the host mammalian subjects.

“Treating” a disease refers to reducing one or more symptoms (such as objective, subjective, pathological, clinical, sub-clinical, etc.) of the disease. Objective symptoms are exemplified by tumor size, blood or urine glucose levels, body weight, etc. Subjective symptoms are exemplified by pain, fatigue, etc.

The invention's compositions may be administered prophylactically (i.e., before the observation of disease symptoms) and/or therapeutically (i.e., after the observation of disease symptoms). Administration also may be concomitant with (i.e., at the same time as, or during) manifestation of one or more disease symptoms. Also, the invention's compositions may be administered before, concomitantly with, and/or after administration of another type of drug or therapeutic procedure (e.g., surgery). Methods of administering the invention's compositions include, without limitation, administration in parenteral, oral, intraperitoneal, intranasal, topical and sublingual forms. Parenteral routes of administration include, for example, subcutaneous, intravenous, intramuscular, intrasternal injection, and infusion routes.

The invention's compositions are typically administered in a therapeutic amount. The terms “therapeutic amount,” “pharmaceutically effective amount,” “therapeutically effective amount,” and “biologically effective amount,” are used interchangeably herein to refer to an amount that is sufficient to achieve a desired result, whether quantitative or qualitative. In particular, a pharmaceutically effective amount is that amount that results in the reduction, delay, and/or elimination of undesirable effects (such as pathological, clinical, biochemical and the like) that are associated with disease. For example, a “therapeutic amount that reduces cancer” is an amount that reduces, delays, and/or eliminates one or more symptoms of cancer.

For example, specific “dosages” of a ““therapeutic amount” will depend on the route of administration, the type of subject being treated, and the physical characteristics of the specific subject under consideration. These factors and their relationship to determining this amount are well known to skilled practitioners in the medical, veterinary, and other related arts. This amount and the method of administration can be tailored to achieve optimal efficacy but will depend on such factors as weight, diet, concurrent medication and other factors, which those skilled in the art will recognize. The dosage amount and frequency are selected to create an effective level of the compound without substantially harmful effects.

Depending on the type and severity of the disease, the invention's compositions are administered at an initial candidate dosage, by one or more separate administrations, or by continuous infusion. For repeated administrations over several days or longer, depending on the condition, the treatment is repeated until a desired suppression of disease symptoms occurs.

In applications where the administered mutant protein is an amino acid degrading enzyme, it is preferred that the administered amount of the enzyme is effective to lower the concentration of the enzyme's substrate in a tissue (e.g., blood, serum, plasma, etc.) of the subject as compared to the concentration of the enzyme's substrate in the absence of administration of the mutant protein

For example, in some embodiments for prokaryotic PAL therapy, the PAL therapy is not continuous, but rather PAL is administered on a daily basis until the plasma phenylalanine concentration of the subject is decreased to a range from below the level of detection to between about 20 μM to 60 μM, preferably less than about 20 μM, and even more preferably less than about 10 μM, using standard detection methods well known in the art. Preferably, wherein the plasma phenylalanine concentration of the subject is monitored on a daily basis and the PAL is administered when a 10%-20% increase in plasma phenylalanine concentration is observed. In yet other preferred embodiments, doses are delivered once weekly. The invention contemplates doses of at least 0.001 mg/kg, 0.005 mg/kg, 0.01 mg/kg, 0.05 mg/kg, and may range up to 0.1 mg/kg, 0.5 mg/kg, 1.0 mg/kg, 5.0 mg/kg, 12 mg/kg or higher per week. A preferred dose is 1 mg/kg/week, a more preferred dose is 0.1 mg/kg/week, and even more preferred dose is 0.01 mg/kg/week (Kakkis et al., U.S. Pat. No. 7,790,433).

The methods of the present invention can be practiced in vitro, in vivo, or ex vivo.

In particular embodiments, the protein is heterologous to the subject, e.g., L-asparaginase II (E. coli) wild-type sequence (SEQ ID NO:03), Arginine Deiminase (Mycoplasma arginini) wild-type amino acid sequence (SEQ ID NO:05), L-methioninase (Pseudomonas putida) wild-type amino acid sequence (SEQ ID NO:06), Phenylalanine ammonia lyase (PAL) (Anabeaena variabilis) wild-type amino acid sequence (SEQ ID NO:07), Urate Oxidase (Aspergillus flavus) (SEQ ID NO:08). Ecotin (E. coli) wild-type amino acid sequence (SEQ ID NO:09), and glutamine deaminase (Burkholderia xenovorans) wild-type amino acid sequence (SEQ ID NO:10).

A “subject” that may benefit from the invention's methods includes any mammal, such as humans, non-human primates, murines, ovines, bovines, ruminants, lagomorphs, porcines, caprines, equines, canines, felines, ayes, etc.). Thus, mammalian subjects are exemplified by mouse, rat, guinea pig, hamster, ferret and chinchilla.

In some embodiments, the subject has, or is at risk of having, a disease, such as acute lymphoblastic leukemia (ALL) (treated with L-asparaginase mutants), hepatocellular carcinomas (treated with arginine deiminase mutants), central nervous system (CNS) cancers (treated with L-methioninase mutants), phenylketonuria (PKU) (treated with phenylalanine ammonia lyase (PAL) mutants), cancer (treated with ecotin mutants and/or phenylalanine ammonia lyase (PAL) mutants), and tumor lysis syndrome (treated with urate oxidase mutants).

In particular embodiments wherein the disease to be treated with the invention's methods is cancer, it may be desirable to further detect reduced proliferation of cancer cells in the subject following administration of the mutant protein of interest compared to prior to the administration.

### EXPERIMENTAL

The following examples serve to illustrate certain embodiments and aspects of the present invention and are not to be construed as limiting the scope thereof.

### Example 1

### Materials & Methods

### FACS Screening:

M9 medium supplemented with 0.4% glucose, 3.5 μg/mL thiamine, 1 mM MgSO₄, 0.1 mM CaCl₂, 160 μg/mL of the amino acids L-Asp and L-Tyr, 80 μg/mL of the 18 remaining amino acids, 30 μg/mL kanamycin, and 200 μg/mL ampicillin was inoculated with a frozen aliquot of E. coli JC1 transformed with pQE80L-GFP (11.3.3) (53) and either a library or a single mutant. Cultures were grown at 37° C. to an A₆₀₀=0.9-1.1, harvested by centrifugation (6000×g, 4 C, 6 min), and washed twice with cold 0.9% NaCl. The cell pellets were resuspended in supplemented M9 medium containing 19 amino acids (no L-Asp, Tyr at 160 μg/mL, remaining amino acids at 80 μg/mL). GFP expression was induced following the media shift by addition of isopropyl-1-thio-β-D-galactopyranoside (IPTG) to a final concentration of 1 mM. After 1 hr induction at 37° C., the cells were harvested by centrifugation (6000×g, 4° C., 6 min), washed twice with PBS, and resuspended in PBS to a final A₆₀₀˜0.05-0.1 for flow cytometric analysis and cell sorting.

Flow cytometric analyses were performed with a FACSAria (BD Biosciences) using a 488 nm solid-state laser for excitation and a 530/30 band pass filter for detection. The throughput rate of cells was adjusted to 4,000-5,000 events per second and ˜10⁷ cells were sorted each round in single cell mode except for the initial sort of each library which was done in purity mode. A gate in the fluorescence channel was set to recover the 4-5% most highly fluorescent cells, while additional gates were set based on both the forward- and side-scatter channels to exclude sorting non-single cell events. The sorted cells were collected in 0.5 mL of 2×YT medium and then plated onto 2×YT medium supplemented with 30 μg/mL kanamycin and 200 μg/mL ampicillin. Following overnight growth at 30° C., the clones were pooled and stored in 15% glycerol at −80° C. in aliquots.

### Transgenic Mice:

HLA-DR4 (DRB1*0401) transgenic mice were generated as described previously (54) and bred under specific pathogen-free conditions at the University of Texas at San Antonio. Transgenic mice were injected at 6-10 weeks of age with the antigen in Complete Freund's Adjuvant (CFA). WT EcAII and variant 3.1.E2 were purified and treated for endotoxin reduction (See below Methods). CFA was prepared by mixing Mycobacterium tuberculosis H37RA (Difco Laboratories, Detroit, Mich.) at 5 mg/mL into IFA. Antigens were mixed with the adjuvant to yield a 2 mg protein/mL emulsion, of which 50 μL was injected subcutaneously as specified. Ten days later, popliteal and inguinal lymph nodes (LN) were removed and single cell suspensions were adjusted to 5×10⁶ cells/mL in HL-1 media (BioWhittaker, Gaithersburg, Md.). Serum was obtained by terminal cardiac puncture. All animal care and experimental procedures were conducted according to guidelines of the Institutional Care and Use Committee (IACUC) at the University of Texas at San Antonio.

### Strains and Plasmids:

Chromosomal gene deletions were performed using the λ-red recombinase system (5). The asparaginase genes ansA, ansB, iaaA, the aspartate aminotransferase gene aspC, and the tyrosine aminotransferase gene tyrB were deleted from the chromosome of E. coli MC1061 (F⁻ Δ(ara-leu)7697 [araD139]_{B/r} Δ(codB-lacI)3 galK16 galE15 λ⁻e14⁻ mcrA0 relA1 rpsL150(strR) spoT1 mcrB1 hsdR2(r⁻m⁺)) resulting in E. coli JC1. Primers used for the deletion of each gene are found in Table 3. Briefly, each primer pair was used to amplify a PCR fragment containing the kanamycin resistance cassette of pKD13. Subsequently, the linear PCR product was used to replace the entire ORF of the targeted gene on the MC1061 chromosome. Colonies containing the correct gene deletions were transformed with the FLP recombinase plasmid pCP20 to remove the kanamycin resistance marker, and the pCP20 was then cured from the resulting strain as described previously (5). The genes ansA, ansB, and iaaA were also deleted from the E. coli strain BL21 (DE3) (F⁻ ompT gal dcm lon hsdS_{B}(r_{B}⁻ m_{B}⁻) λ(DE3 [ladI lacUV5-T7 gene 1 ind1 sam7 nin5]) chromosome, resulting in E. coli JC2, used to express the EcAII variants. Where necessary, gene deletions were transferred to recipient strains via P1 transduction.

All plasmids and primers used in this work are described in Table 2 and Table 3, respectively.

TABLE 2
Plasmids used in this work

| | | Reference or |
|---|---|---|
| Plasmids | Relevant Characteristics | Source |
| pET-28a | Kan^{R}, T7 promoter | Novagen |
| pHisEcAII | Kan^{R}, T7 promoter, | This study |
| | encodes His6x-EcAII | |
| pET-26b | Kan^{R}, T7 promoter | Novagen |
| pPelbHisEcAII | Kan^{R}, T7 promoter, | This study |
| | encodes sspelb-His6x-EcAII | |
| pPelbHisT12A | Kan^{R}, T7 promoter, | This study |
| | encodes sspelb-His6x-EcAII(T12A) | |
| pCTK | Kan^{R}, tet promoter, CloDF ori | This study |
| pASK75 | Amp^{R}, tet promoter | 3 |
| pCDF-1b | Sp^{R}, T7 promoter, CloDF ori | Novagen |
| pCTK-EcAII | encodes sspelb-His6x-EcAII in pCTK | This study |
| pCTK-T12A | encodes sspelb-His6x-EcAII(T12A) | This study |
| | in pCTK | |
| pCTK-G57A | encodes sspelb-His6x-EcAII(G57A) | This study |
| | in pCTK | |
| pCTK-G57V | encodes sspelb-His6x-EcAII(G57V) | This study |
| | in pCTK | |
| pCTK-G57L | encodes sspelb-His6x-EcAII(G57L) | This study |
| | in pCTK | |
| p28pelHisEcAII | encodes sspelb-His6x-EcAII in | This study |
| | pET-28a | |
| p28pelHis1.1.C4 | encodes sspelb-His6x-1.1.C4 in | This study |
| | pET-28a | |
| p28pelHis2.2.G10 | encodes sspelb-His6x-2.2.G10 in | This study |
| | pET-28a | |
| p28pelHis3.1.E2 | encodes sspelb-His6x-3.1.E2 in | This study |
| | pET-28a | |
| pQE80L- | Amp^{R}, lac promoter, encodes | 4 |
| GFP(11.3.3) | GFP(11.3.3) | |

TABLE 3
primers used in this work

| Primer Name | Nucleotide Sequence (5′ → 3′) |
|---|---|
| ansBFor | GTGCAGCACATATGTTACCCAATATCACCCA |
| ansBRev | GGCGGGATCCTTAGTACTGATTGAAGA |
| T12AFor | GTGCAGCACATATGTTACCCAATATCACCATTTTAGCAACCGGCGGGGCCA |
| T7term | GCTAGTTATTGCTCAGCGG |
| tetFor | CTAGCTAGTCTAGAGCGGAGCCTATGGAAAAACGC |
| tetRev | CATGCCATGGCACTTTTCTCTATCACTGATAGGG |
| KanFor | GGGCCGGCAGGCGCTCCATTGCCCAGTCGGCTAAGGGATTTTGGTCATGAAC |
| KanRev | GCGTAGCGACCGAGTGAGCTAGCTTATTAGAAAAACTCATCGAGCATC |
| 5′ ansA KO | CCTCACGTATATACTTTTGCTCTTTCGATATCATTCATATCAATATCATGATTCCGGGGATCCGTCGACC |
| 3′ ansA KO | ACAGGGCGCGAGGGGGCATTACAGTCTCCTTAATCATCCGGCGTCAGTTCTGTAGGCTGGAGCTGCTTCG |
| 5′ andB KO | CAGAGCTAAGGGATAATGCGTAGCGTTCACGTAACTGGAGGAATGAAATGATTCCGGGGATCCGTCGACC |
| 3′ andB KO | AGCCCCGGCACGATACCGGGGCGAGGCGATTAGTACTGATTGAAGATCTGTGTAGGCTGGAGCTGCTTCG |
| 5′ laaA KO | TGATATTTATAGCAAAAGTGGCGAACCACCCTTAATGGACGAATACTATGATTCCGGGGATCCGTCGACC |
| 3′ laaA KO | CCGCCAGCACATTACCGGCATCAAGTTCATCACTGTGTGGCAACGGTGTCTGTAGGCTGGAGCTGCTTCG |
| 5′ aspC KO | TACCCTGATAGCGGACTTCCCTTCTGTAACCATAATGGAACCTCGTCATGATTCCGGGGATCCGTCGACC |
| 3′ aspC KO | TTTTCAGCGGGCTTCATTGTTTTTAATGCTTACAGCACTGCCACAATCGCTGTAGGCTGGAGCTGCTTCG |
| 5′ tyrB KO | GTTTATTGTGTTTTAACCACCTGCCCGTAAACCTGGAGAACCATCGCGTGATTCCGGGGATCCGTCGACC |
| 3′ tyrB KO | GCTGGGTAGCTCCAGCCTGCTTTCCTGCATTACATCACCGCAGCAAACGCTGTAGGCTGGAGCTGCTTCG |
| 5′ MSSA-NNS | GGTCGGCGCANNSCGTCCGNNSACGNNSATGAGCNNSGACGGTCCATTCAACCTG |
| 3′ MSSA-NNS | CAGGTTGAATGGACCGTCSNNGCTCATSNNCGTSNNCGGACGSNNTGCGCCGACC |
| 5′ INAS-NNS | CTGCCGAAAGTCGGCNNSGTTTATNNSTACNNSAACGCANNSGATCTTCCGGCTAAAGCACTG |
| 3′ INAS-NNS | CAGTGCTTTAGCCGGAAGATCSNNTGCGTTSNNGTASNNATAAACSNNGCCGACTTTCGGCAG |
| 5′ VQAQ-NNS | CCGCAAAAAGCGCGCNNSCTGCTGNNSCTGNNSCTGACGNNSACCAAAGATCCGCAGCAG |
| 3′ VQAQ-NNS | CTGCTGCGGATCTTTGGTSNNCGTCAGSNNCAGSNNCAGCAGSNNGCGCGCTTTTTGCGG |
| pCTKFor | CGATCAAACCACCTCCCCAGGTGGTTTTTTCGTTTACAGGGC |
| 5′ G57A | GGTAGTGAATATCGCGTCCCAGGACATGAACG |
| 5′ G57V | GGTAGTGAATATCGTGTCCCAGGACATGAACG |
| 5′ G57L | GGTAGTGAATATCCTGTCCCAGGACATGAACG |
| 3′ g57mp | GGTGACGTCACGGCCATCAAGCACGGTGTCATTCATC |

PCR reactions were carried out using Vent DNA polymerase (New England Biolabs) and oligonucleotides were synthesized by Integrated DNA Technologies. The ansB gene (mature sequence only) was amplified from the genomic DNA of E. coli K12 using the primers ansBFor/ansBRrev, digested with NdeI-BamHI, and cloned into pET-28a to generate plasmid pHisEcAII Subsequently, plasmid pPelBHisEcAII was generated through subcloning the NcoI-BamHI digested fragment from pHisEcAII into pET-26b. In addition, a plasmid coding EcAII-T12A was generated by PCR using the pPelBHisEcAII plasmid as template and the primer pair T12AFor/T7 term, resulting in plasmid pPelBHisT12A.

To construct vector pCTK, firstly, the tet promoter region from vector pAS K75 was amplified using the primers tetFor/tetRev, digested with XbaI-NcoI, and ligated into pCDF-1b.

Next, the kanamycin resistance cassette from vector pET-28a was amplified using the primers KanFor/KanRev, digested with BglI-BmtI, and then cloned into pCDF-1b as well, ultimately generating the final pCTK vector.

Plasmids pCTK-EcAII and pCTK-T12A were generated by subcloning the NcoI-NotI digested fragments of pPelBHisEcAII and pPelBHisT12A respectively, into vector pCTK. Plasmids pCTK-G57A, pCTK-G57V, and pCTK-G57L were constructed using a 2-step protocol based on the QuickChange methodology. In the first step, megaprimers for each G57 mutant were amplified by PCR from the plasmid pCTK-EcAII using the primer pair 5′-G57X (X=A, V, L)/3′-G57 mp. In the second step, the megaprimer was used in place of outside primers in a PCR reaction again using plasmid pCTK-EcAII as the template with the following cycling parameters: 95° C.-2 min, 16 cycles of 95° C.-30 s, 55° C.-1 min, 72° C.-10 min, and a final polishing step at 72° C.-15 min. Each product was then digested with DpnI for 1 hr at 37° C. to eliminate the initial template plasmid.

### In Silico Identification and Characterization of EcaII T-Cell Epitopes:

The primary sequence corresponding to the mature region of EcAII was screened for putative T-cell epitopes using the IEDB consensus prediction method. The 326 amino acid sequence was parsed into overlapping 15-mer peptide fragments and within each fragment, a 9-mer core region was identified and scored for predicted binding by a consensus percentile rank (CPR) in which a lower score (in arbitrary units) was indicative of a higher predicted binding affinity. Because the consensus method scoring is based on the outputs of individual MHC-II binding prediction methods, multiple 9mer cores were identified in some 15mer fragments. In these instances, the 9mer core selected was the one predicted by TEPITOPE (Sturniolo) (6), which served as the basis for ProPred (7)—the most accurate algorithm for epitope core identification among those evaluated by the developers of the IEDB consensus method (8). Binding was further evaluated for 7 additional HLA-DR alleles which when taken with DRB1*0401 cover nearly 95% of human populations worldwide (9). Three 9mer core regions that were scored with a consensus percentile rank (CPR) falling within the lowest 10% of the parsed peptide fragments as determined for binding to DRB1*0401 (CPR<2) and that further received equivalently low scores for at least one other DRB 1 allele were selected for neutral drift combinatorial mutagenesis.

### Library Construction:

Oligonucleotides encoding degenerate NNS (N is A, T, G, C; S is G, C) codons at the sites corresponding to residues in positions P1, P4, P6, and P9 of each of the three 9mer core sequences chosen for mutagenesis were used for library construction and can be found in Table 3. For the first library, PCR with Vent DNA polymerase and pCTK-EcAII as template was carried out to generate two fragments from the primer pairs pCTKFor/3′ MSSA-NNS and 5′ MSSA-NNS/T7term respectively. The DNA fragments obtained from these PCRs were electrophoresed and purified using a QIAGEN gel purification kit. Equimolar quantities of the two fragments were then mixed and subjected to overlap-extension PCR using the primers pCTKFor/T7term. The resulting 1.5-kb PCR product was digested with NcoI-NotI and ligated into pCTK-T12A digested with the same enzymes. The ligation mixture was then transformed into electrocompetent E. coli JC1 encoding plasmid pQE80L-GFP(11.3.3) (4), yielding ˜10⁷ individual transformants. The clones were pooled and stored in 15% glycerol at −80 C in aliquots. The second and third libraries were constructed analogously, using the internal primers 5′ INAS-NNS/3′ INAS-NNS or 5′ VQAQ-NNS/3′ VQAQ-NNS in place of 5′ MSSA-NNS/3′ MSSA-NNS respectively.

### Colorimetric Asparaginase Activity Assay:

A colorimetric asparaginase assay using L-Aspartic Acid β-hydroxomate (AHA) (10) was used to isolate active asparaginase clones from the final FACS-sorted population of each library. Following the final round of sorting, the polyclonal gene cassette of the collected population was amplified using primers pCTKFor/T7term, digested with NcoI-NotI, and subcloned into pET-28a digested with the same restriction enzymes. The ligation mixture was transformed into electrocompetent E. coli JC2 and single colonies were used to inoculate 90 μL 2×YT supplemented with 30 μg/mL kanamycin over two 96-well plates. Following 2 hr incubation (with shaking at 350 rpm) at 37° C., protein expression was induced by adding an additional 90 μL 2×YT (30 μg/mL kanamycin, 1 mM IPTG) to each well. After 3 hr induction at 37° C., 120 μL from each well was transferred to a fresh 96-well plate and cells were harvested by centrifugation (3500×g, 4 C, 10 min). The cell pellets were then resuspended in 120 μL B-PER Bacterial Protein Extraction Reagent (Thermo Scientific), incubated at 25° C. for 20 min with shaking at 350 rpm, and subsequently pelleted (3500×g, 4 C, 15 min). The resulting supernatants were transferred to a Ni2+-NTA HisSorb Plate (QIAGEN) and stored at 4° C. overnight. After decanting the supernatant and rinsing twice with wash buffer (50 mM Tris-HCl, 100 mM NaCl, 25 mM imidazole, pH 8), 50 μL of 10 mM AHA in activity buffer (50 mM Tris-HCl, 100 mM NaCl, pH 7.4) was added to each well. Following incubation with substrate at 25° C. for 20 min, 50 μL color reagent (2% 8-hydroxyquinoline in ethanol/1 M Na₂CO₃, 1:3 by volume) was added to each well and the plate was covered, heated in a 100° C. oven for 90 s, and allowed to cool at 4° C. for 15 min. Activity within each well was quantitated by measuring the absorbance at 705 nm (Synergy HT Fluorescent Platereader, BioTek).

### Expression and Purification of EcAII Variants:

E. coli JC2 harboring pET-28a encoding either WT EcAII or an isolated mutant EcAII (p28pelHisEcAII, p28pelHis1.1.C4, p28pelHis2.2.G10, and p28pelHis3.1.E2) were cultured overnight at 37° C. in 2×YT medium supplemented with 30 μg/mL kanamycin and used to inoculate 250 mL fresh medium (1:100). When the A₆₀₀ reached 0.5-0.7, the cells were transferred to 25° C. and allowed to equilibrate for 20 min, at which point the culture was supplemented with IPTG to a final concentration of 1 mM to induce protein expression. After 16 hr of incubation at 25° C., the cells were harvested by centrifugation at 10,000×g for 10 min. The cell pellet was resuspended in binding buffer (50 mM Tris-HCl, 100 mM NaCl, 10 mM imidazole, pH 8), lysed by three passes through a French pressure cell, and subsequently pelleted at 40,000×g for 45 min. The resulting supernatant (soluble fraction) was decanted, diluted 1:1 in binding buffer and mixed with 0.5 mL of pre-equilibrated nickel-nitrilotriacetic acid (Ni²⁺⁻NTA) resin. After incubation for 90 min at 4° C. with gentle rotation, the solution was applied to a 5 mL polypropylene column. The resin was then washed with 25 bed volumes binding buffer and 25 bed volumes wash buffer (50 mM Tris-HCl, 100 mM NaCl, 25 mM imidazole, pH8) before the resin was incubated with 4 mL elution buffer (50 mM Tris-HCl, 100 mM NaCl, 250 mM imidazole, pH8) for 10 min and collected drop-wise. The eluted fractions were concentrated using an Amicon Ultra 10K MWCO filter and purified into PBS by gel filtration on a Superdex 200 column (Amersham Pharmacia).

### Determination of Kinetic Parameters:

The kinetics of L-Asn hydrolysis were determined with freshly purified enzyme as described previously (11). Briefly, reactions of each asparaginase variant (10-20 nM enzyme) with L-Asn (0 to 5×K_{M}) were carried out at 37° C. in 50 mM Tris-HCl, 100 mM NaCl (pH 7.4) in a total volume of 100 μL, and were subsequently quenched with 5 μL of 12% (w/v) trichloroacetic acid. An aliquot of the quenched reaction mixture was then mixed with a molar excess (relative to substrate) of o-phtalaldehyde (OPA) reagent and brought to a final volume of 100 μL, with borate buffer. The resulting solutions were analyzed by HPLC using an Agilent ZORBAX Eclipse AAA Column (C18 reverse phase, 5 μm, 4.6 mm×150 mm). All reactions were conducted at least in triplicate and the observed rates were fit to the Michaelis-Menten equation using Kaliedagraph (Synergy).

### Asparaginase Serum Stability:

Approximately 250 μg of WT EcAII or variant 3.1.E2 was mixed with 1 mL pooled human serum (Innovative Research) and incubated at 37° C. At various time points, a 15 μL aliquot from each sample was removed and used to set up triplicate reactions in which 5 μL per aliquot was added to 50 μL 10 mM AHA in activity buffer in a microtiter plate. After allowing the reaction to proceed for 5 min at room temperature, 50 μL color reagent was added to each well, the plate was covered, heated in a 100° C. oven for 90 s, and then allowed to cool at 4° C. for 15 min. Activity within each well was quantitated by measuring the absorbance at 705 nm (Synergy HT Fluorescent Platereader, BioTek) and the average values for each enzyme-containing sample were then normalized by subtracting the average value measured for the control sample at the same time point.

Antigen Preparation and EcAII peptide Library:

Endotoxin contamination of purified enzymes was reduced by a previously described phase separation technique using the detergent Triton X-114 (12). Following 6-8 phase separation cycles, protein was buffer exchanged against sterile, commercially purchased 1×PBS (Gibco) using an Amicon Ultra 10K MWCO filter to remove any detergent that may have persisted within the solution. Enzyme preparations treated by this procedure retained normal activity. The endotoxin levels of the enzyme were determined by Limulus Amebocyte Lysate (LAL) assay and observed to be <7 endotoxin units/100 μg protein. A collection of 32 overlapping 20mer EcAII peptides (Table 4), staggered by 10 amino acids and spanning the entire primary sequence, were synthesized by GenScript.

TABLE 4
Overlapping synthetic peptides of WT
EcAII for T-cel activation assays

| Peptide Name | Peptide Sequence |
|---|---|
| WTp1-20 | MLPNITILATGGTIAGGGDS |
| WTp11-30 | GGTIAGGGDSATKSNYTVGK |
| WTp21-40 | ATKSNYTVGKVGVENLVNAV |
| WTp31-50 | VGVENLVNAVPQLKDIANVK |
| WTp41-60 | PQLKDIANVKGEQVVNIGSQ |
| WTp51-70 | GEQVVNIGSQDMNDNVWLTL |
| WTp61-80 | DMNDNVWLTLAKKINTDCDK |
| WTp71-90 | AKKINTDCDKTDGFVITHGT |
| WTp81-100 | TDGFVITHGTDTMEETAYFL |
| WTp91-110 | DTMEETAYFLDLTVKCDKPV |
| WTp101-120 | DLTVKCDKPVVMVGAMRPST |
| WTp111-130 | VMVGAMRPSTSMSADGPFNL |
| WTp121-140 | SMSADGPFNLYNAVVTAADK |
| WTp131-150 | YNAVVTAADKASANRGVLVV |
| WTp141-160 | ASANRGVLVVMNDTVLDGRD |
| WTp151-170 | MNDTVLDGRDVTKTNTTDVA |
| WTp161-180 | VTKTNTTDVATFKSVNYGPL |
| WTp171-190 | TFKSVNYGPLGYIHNGKIDY |
| WTp181-200 | GYIHNGKIDYQRTPARKHTS |
| WTp191-210 | QRTPARKHTSDTPFDVSKLN |
| WTp201-220 | DTPFDVSKLNELPKVGIVYN |
| WTp211-230 | ELPKVGIVYNYANASDLPAK |
| WTp221-240 | YANASDLPAKALVDAGTDGI |
| WTp231-250 | ALVDAGYDGIVSAGVGNGNL |
| WTp241-260 | VSAGVGNGNLYKSVFDTLAT |
| WTp251-270 | YKSVFDTLATAAKTGTAVVR |
| WTp261-280 | AAKTGTAVVRSSRVPTGATT |
| WTp271-290 | SSRVOTGATTQDAEVDDAKY |
| WTp281-300 | QDAEVDDAKYGFVASGTLNP |
| WTp291-310 | GFVASGTLNPQKARVLLQLA |
| WTp301-320 | QKARVLLQLALTQTKDPQQI |
| WTp311-326 | LTQTKDPQQIQQIFNQY |

An additional set of peptides corresponding to sequences containing the engineered mutations in 3.1.E2 were also synthesized (Abgent).

### Cytokine Measurements by ELISPOT and Computer-Assisted ELISPOT Image Analysis:

Cytokine ELISPOT assays were performed as described previously (13). Briefly, ELISPOT plates (Multiscreen IP, Millipore, Billerica, Mass.) were coated overnight with 2 μg/mL IFN-γ-specific capture antibody (AN-18; eBioscience, San Diego, Calif.) diluted in PBS. The plates were blocked with 1% BSA in PBS for 1 hr at room temperature and then washed four times with PBS. LN cell suspensions were plated at 5×10⁵ cells/well with either whole antigen or with EcAII overlapping peptides and incubated at 37° C. for 24 hr. Note that cells were plated with the whole antigen or overlapping peptides corresponding to the EcAII variant used to immunize the mouse from which they were isolated. Subsequently, the cells were removed by washing three times with PBS and four times with PBS/Tween, and IFN-γ-specific biotinylated detection Ab (R4-6A2; 0.5 μg/mL, eBioscience) was added and incubated overnight. The plate-bound secondary antibody was then incubated with streptavidin-alkaline phosphatase (Dako, Carpinteria, Calif.), and cytokine spots were visualized by 5-bromo-4-chloro-3-indolyl phosphate/NBT phosphatase substrate (KPL, Gaithersburg, Md.). Image analysis of ELISPOT assays was performed on a Series 2 Immunspot analyzer and software (Cellular Technology, Cleveland, Ohio) as described previously. In brief, digitized images of individual wells of the ELISPOT plates were analyzed for cytokine spots based on the comparison of experimental (containing T-cells and APC with Ag or peptide) and control wells (T-cells and APC without Ag or peptide). Following the separation of spots that were touched or partially overdeveloped, nonspecific noise was gated out by applying spot size and circularity analysis as additional criteria. Spots that fell within the accepted criteria were highlighted and counted.

### Detection of Antigen-Specific Antibody Titer by ELISA:

Serum was obtained by terminal cardiac puncture from mice immunized by subcutaneous injection with either WT EcAII or 3.1.E2, as described in the main text method ‘Transgenic Mice’. Microtiter plates (eBioscience 44-2504-21) were coated overnight at 4° C. with 1 μg of antigen (WT EcAII or 3.1.E2) in PBS and blocked for an additional 1 hr at room temperature with 1× assay diluent (eBioscience #00-4202-56). Serial dilutions of sera were added to wells coated with the corresponding immunizing antigen and incubated for 2 hr at room temperature. The plates were washed and incubated with ImmunoPure goat anti-mouse IgG conjugated with horseradish peroxidase for 1 hr at room temperature. The plates were subsequently washed, incubated with tetramethylbenzidine (TMB) substrate for 15 min at room temperature before the reactions were stopped by addition of 2M H₂SO₄. The absorbance was read (450 nm) using an ELISA microplate reader (μQuant; Biotek Instruments, Winooski, Vt.). End-point titers were calculated by using an absorbance corresponding to a control well (PBS substituted for sera) as the cutoff value.

### Example 2

### Development and Validation of Screen

To develop a screen for EcAII, the inventors first constructed E. coli JC1 [MC1061 ΔaspCΔtyrBΔansAΔansBΔiaaA] in which the genes required for L-Asp biosynthesis (aspC, tyrB) and the three genes required for endogenous L-asparaginase enzymes were deleted. JC1 cells expressing a low level of recombinant EcAII formed normal size colonies when plated on minimal media plates with 19 amino acids (no L-Asp). In contrast, cells without plasmid or expressing the recombinant, inactive, EcAII-T12A point mutant formed pinpoint colonies, presumably because spontaneous hydrolysis of L-Asn provides a basal level of L-Asp for growth. The formation of pinpoint colonies by null mutants and the cross-feeding of L-Asp generated by low activity clones frustrated efforts to select neutral mutants on plates with selective media; in multiple attempts, similar size colonies formed by plating mutagenized enzyme were later found to encode enzymes with dramatically different L-Asn hydrolysis kinetics.

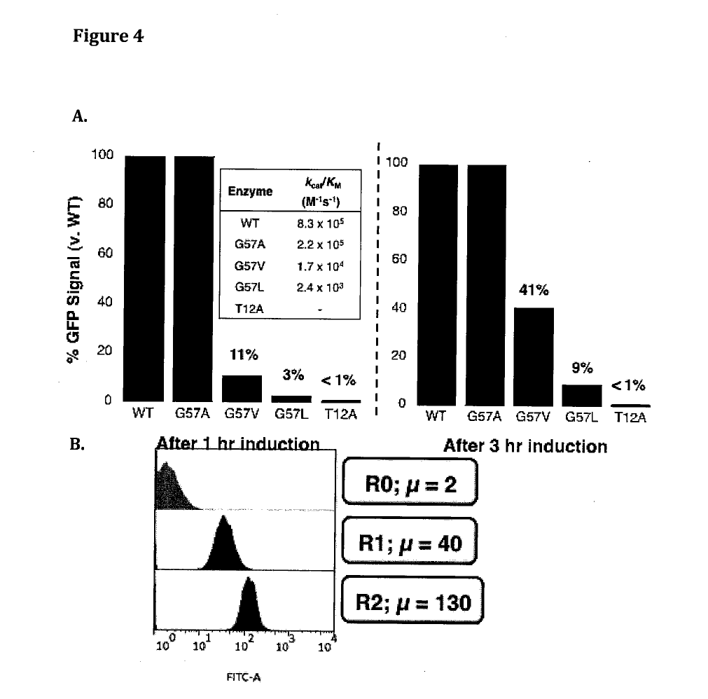

To enable an additional level of quantitation, cells were transformed with a plasmid expressing green fluorescent protein (GFP) under an isopropyl β-D-1-thiogalactopyranoside (IPTG) inducible promoter, grown to late exponential phase in media containing all 20 amino acids, washed and transferred for a short time period to media with 19 amino acids (no L-Asp) and IPTG to induce GFP synthesis. Following the addition of IPTG, GFP synthesis—and hence intracellular fluorescence—was dependent on the availability of L-Asp, which in turn was proportional to EcAII enzymatic activity. Utilizing this assay, the inventorsfound that intracellular GFP fluorescence correlated well with the enzyme activity of a panel of recombinantly expressed EcAII variants displaying up to two orders of magnitude differences in catalytic efficiency. Thus, in contrast to the genetic selection approach described earlier, co-expression of a GFP reporter allowed for the discrimination of clones expressing asparaginases with varying degrees of enzymatic activity. Upon induction of GFP transcription from a strong promoter on a high copy number plasmid, the GFP mRNA accounts for a large fraction of the total cellular mRNA, therefore providing a timeframe during which GFP synthesis correlates well with the rate of L-Asp generation, and thus with the relative asparaginase activity within the cell. For example, cells expressing EcAII-G57V exhibited a nearly 10-fold lower GFP fluorescence relative to cells expressing EcAII-G57A which has approximately 15-fold higher catalytic efficiency (42) (FIG. 4A). While the assay was able to easily eliminate low activity clones, cells expressing enzymes with high catalytic activity, such as EcAII-G57A (k_{cat}/K_{M} (L-aspartic acid β-hydroxomate (AHA))=2.2×10⁵M⁻¹S⁻¹ (42)), exhibited identical GFP fluorescence relative to WT EcAII (k_{cat}/K_{M} (AHA)=8.2×10⁵ M⁻¹S⁻¹ (42)) indicating that the signal saturates for enzymes with k_{cat}/K_{M} within 3 to 4-fold of the WT enzyme catalytic efficiency.

Nonetheless, enzymes with k_{cat}/K_{M} (L-Asn)>10⁶ M⁻¹ s⁻¹ should be more than sufficient for therapeutic purposes given that circulating L-Asn is depleted to negligible levels within minutes following the administration of a therapeutic dose of WT EcAII (43), and remain low for weeks afterwards (32, 43). Therefore, even though enzymes with k_{cat}/K_{M} (L-Asn) up to 3 to 4-fold below that of the WT enzyme might result in marginally slower initial depletion of serum L-Asn, they should not affect the longer term maintenance of low serum L-Asn levels which is the therapeutically relevant parameter.

To validate the enrichment capabilities of the assay, three rounds of cell sorting produced a 6.000-fold enrichment of JC1 cells expressing WT EcAII from an initial mixture containing a 10.000-fold excess of E. coli JC1 expressing EcAII-T12A (FIG. 4B).

### Example 3

### Computational Identification of Putative EcAII T-Cell Epitopes

Putative EcAII T-cell epitopes were identified using the Immune Epitope Database (IEDB) consensus method (16). The protein sequence was parsed into overlapping 15mer peptide fragments (staggered by one residue) and within each fragment, 9-mer core regions were scored for predicted binding first to HLA-DRB1*0401, which shows strong association with childhood ALL in males (44), and then to an additional seven HLA-DR alleles that collectively cover nearly 95% of the human population (45). Three 9-mer core regions scored with a consensus percentile rank (CPR) within the lowest 10% of the parsed peptide fragments for binding to DRB1*0401 (CPR<2); the regions that further showed equivalently low scores for at least one other DRB1 allele were selected for T-cell epitope removal: M₁₁₅RPSTSMSA, I₂₁₆VYNYANAS, and V₃₀₄LLQLALTQ (designated M₁₁₅, I₂₁₆, and V₃₀₄ where these three residues correspond to the respective P1 positions).

### Example 4

### Screening of EcAII Libraries

The P1, P4, P6, and P9 positions, which are most critical for the binding of peptides to the MHC-II binding groove (15), were subjected to saturation mutagenesis using the NNS (N=A, T, G, C; S=G, C) randomization scheme. Randomization and neutral drift screening were carried out sequentially, starting with M₁₁₅ and continuing with I₂₁₆ and finally V₃₀₄ to: (i) ensure complete library coverage for each individual epitope; (ii) evaluate the relative plasticity of different regions of the protein to amino acid substitutions, and (iii) simplify the structural interpretation of any observed changes in the activity of isolated mutants.

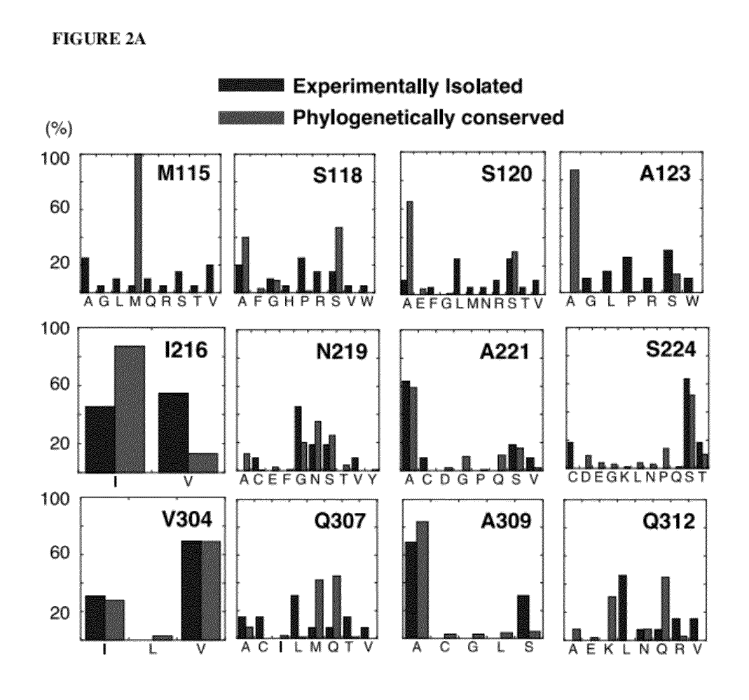

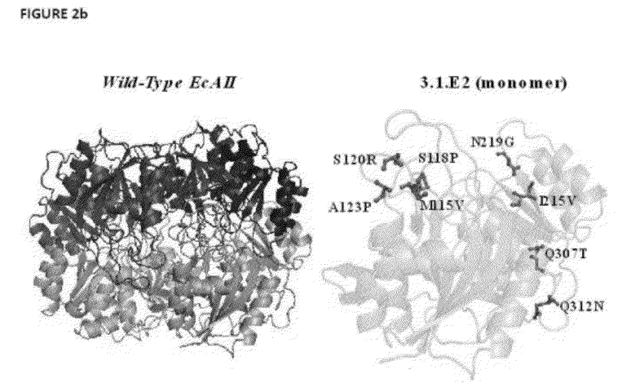

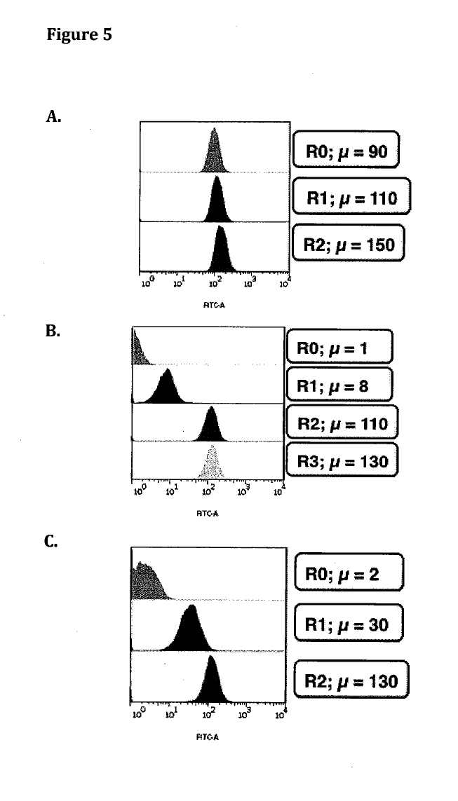

10⁷ transformants of the M₁₁₅ library (predicted theoretical diversity≈10.5⁶) were subjected to 3 rounds of FACS screening until the mean cell fluorescence of the sorted population was comparable to that of cells expressing the WT enzyme (FIG. 5A). In this instance, the high initial fluorescence of the library (μ=90; FIG. 5A) suggested that amino acid substitutions at the targeted sites were generally tolerated. Following the final round of sorting, 120 individual clones selected at random were assayed in microtiter well plates using the colorimetric asparaginase substrate AHA. This secondary screen eliminated inactive clones that might have been inadvertently recovered during FACS enrichment. Active clones were found to display minor variations in AHA hydrolysis rate consistent with the notion that the FACS screen enriches variants with near WT activity. FIG. 2A shows the frequency of amino acid occupancy at M115, S118, S120, and A123. Surprisingly, M115, which is absolutely conserved among the nearly 500 bacterial type II L-asparaginases in the database, could tolerate a variety of non-conservative substitutions. Analogous surprising promiscuity was observed at both S120 and A123, which are also highly conserved phylogenetically. Evaluation of the isolated sequences using the IEDB consensus model revealed that the alteration of M₁₁₅RPSTSMSA to V₁₁₅RPPTRMSP results in over a 20-fold increase in CPR score for the DRB1*0401 allele as well as increases in the CPR scores for 5 other HLA-DR alleles (Table 1).

TABLE 1
Computational prediction of T cell epitopes in wt EcAII and
the 3.1.E2 mutant by IEDB consensus method

Minimum CPR

| HLA | MRPSTSMSA | | IVYNYANAS | | VLLQLALTQ | |
|---|---|---|---|---|---|---|
| Allele | (VRPPTRMSP) | | (VVYGYANAS) | | (VLLTLALTN) | |
| DRB1*0101 | 18.48 | (n/a) | 5.6 | (12.27) | 6.9 | (n/a) |
| DRB1*0301 | 2.29 | (2.24) | 1.07 | (6.71) | 6.71 | (15.89) |
| DRB1*0401 | 1.40 | (30.75) | 0.27 | (1.88) | 0.79 | (2.42) |
| DRB1*0701 | 18.53 | (n/a) | 0.56 | (3.95) | n/a | (18.91) |
| DRB1*0801 | 11 | (9.9) | 5.17 | (9.9) | 6.4 | (9.9) |
| DRB1*1101 | 1.82 | (6.94) | 1.12 | (7.58) | 0.98 | (0.94) |
| DRB1*1301 | 6.36 | (6.41) | 0.38 | (2.93) | 2.40 | (13) |
| DRB1*1501 | 19.91 | (27.58) | 3.14 | (11.83) | 10.89 | (9.32) |
| CPR ≦ 2 | 2 | | 5 | | 2 | |

Minimum consensus percentile rank score (CPR) for the 3 targeted EcAII T-cell epitope core regions across 8 common HLA-DR alleles (See Methods of Example 1).
The ALL-associated DRB1*0401 allele is shown in bold.
Lower scores are indicative of high predicted binding affinity.
Sequence and scores for the 3.1.E2 mutant are shown in parenthesis where mutations relative to wt EcAII are in bold.
n/a: not predicted to bind.

The resulting enzyme variant, EcAII M115V/S118P/S120R/A123P (designated as clone 1.1.C4), having 4 amino acid substitutions, 3 of which were non-conservative, displayed catalytic properties for the hydrolysis of L-Asn (k_{cat}=28 s⁻¹, K_{M}=17 μM, k_{cat}/K_{M}=1.6×10⁶ M⁻¹s⁻¹) nearly identical to those of the parental enzyme with only a 2-fold increase in K_{M}.

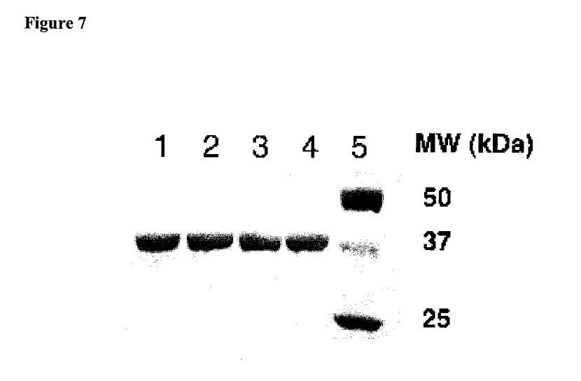

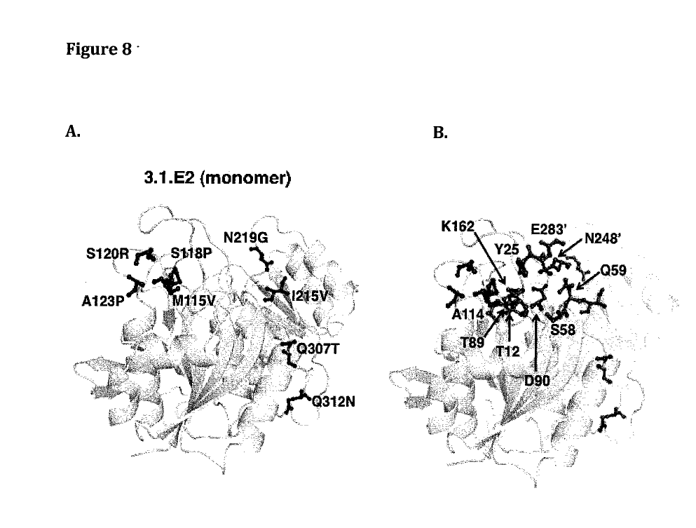

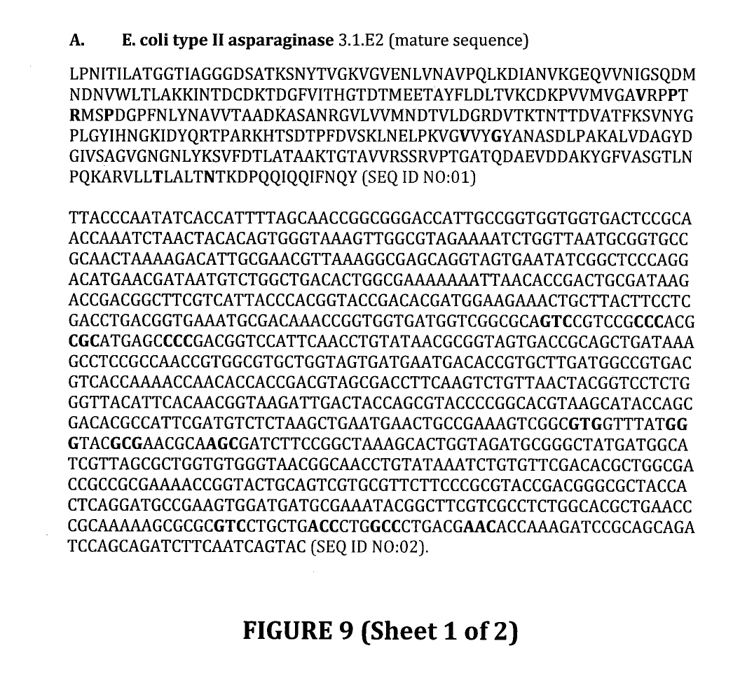

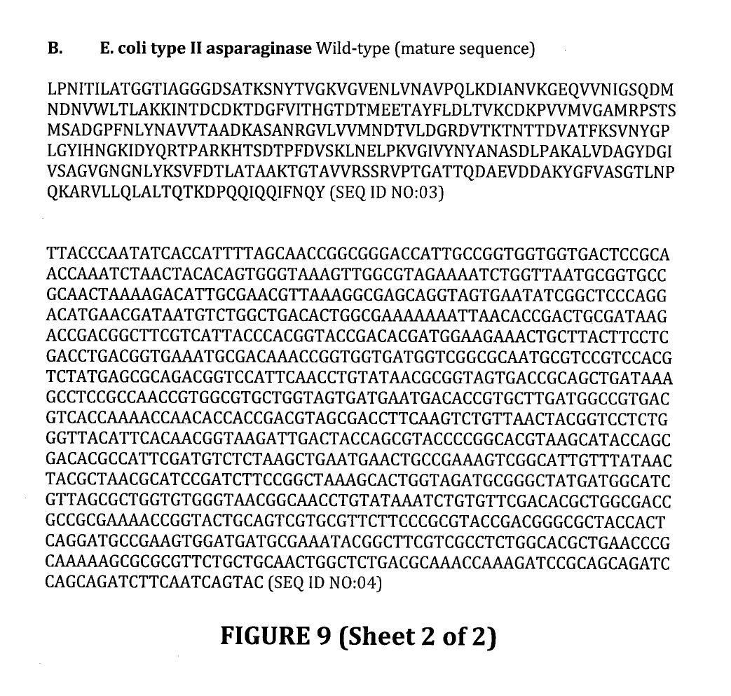

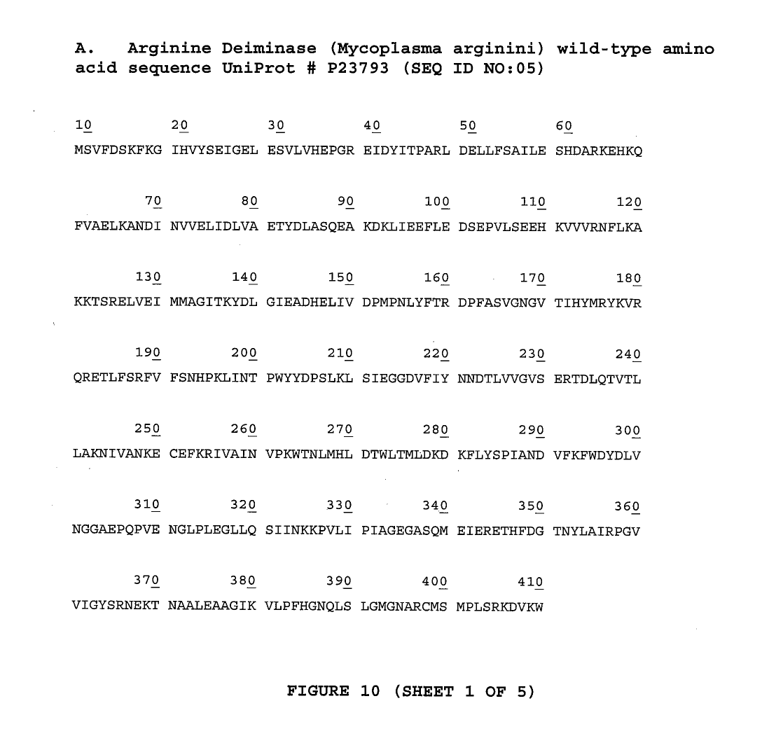

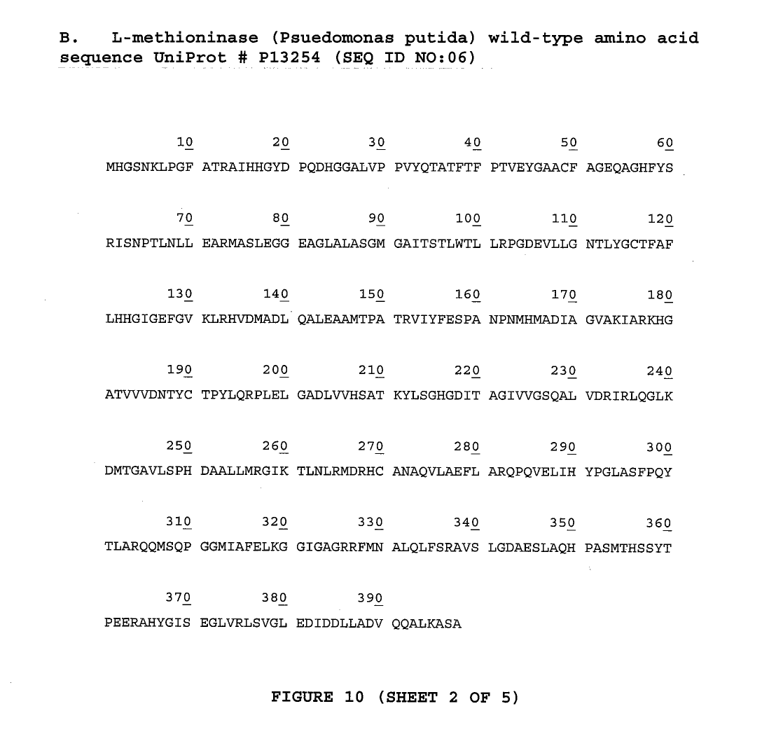

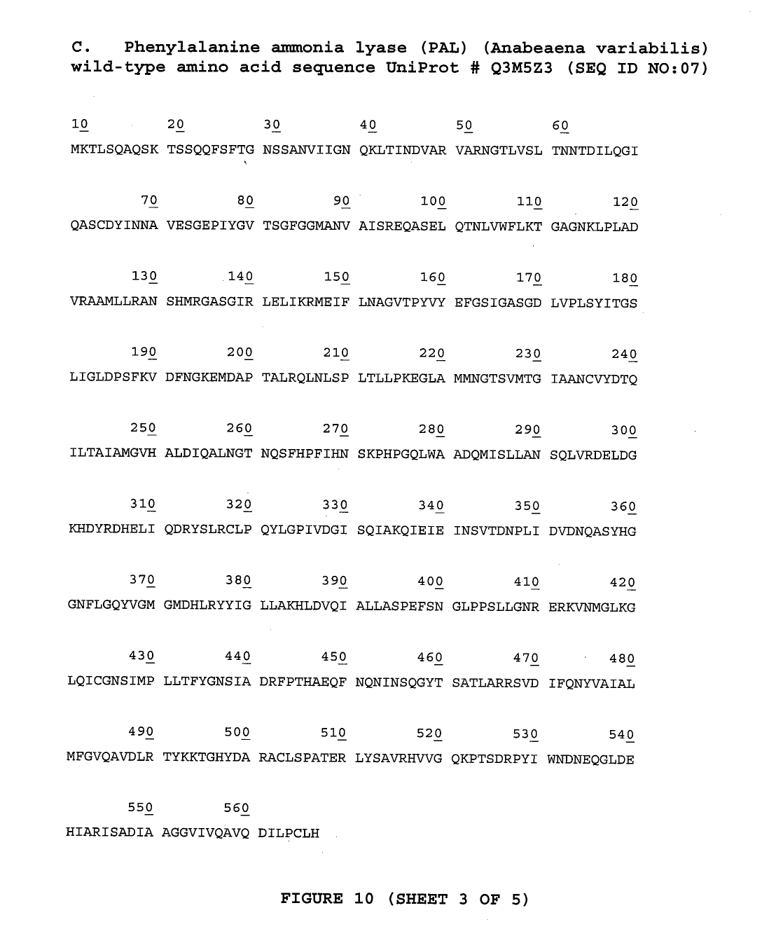

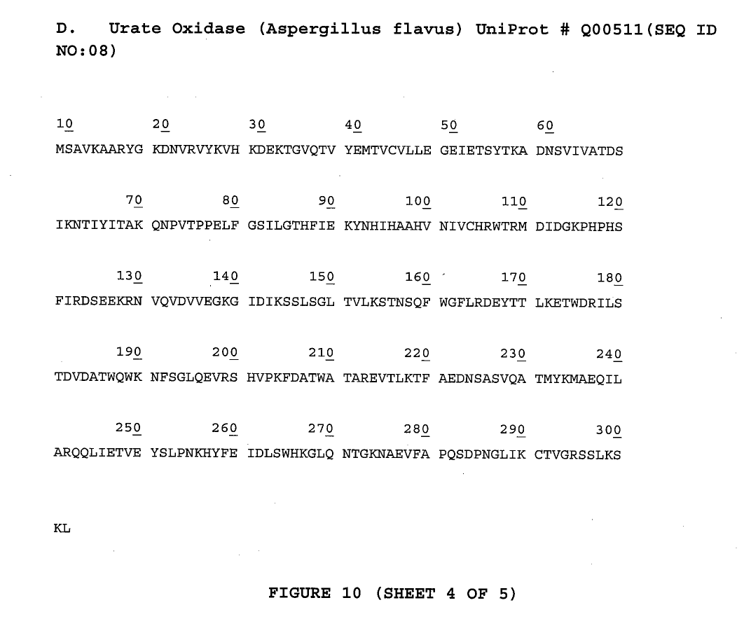

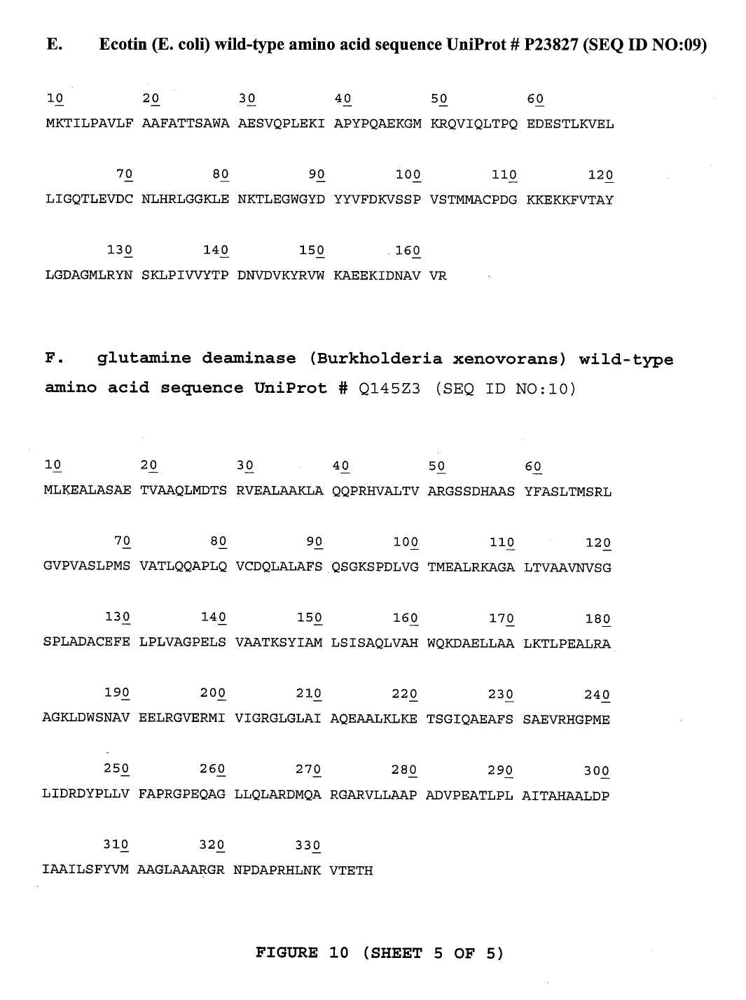

The 1.1.C4 variant was then used as a template to diversify the P1, P4, P6, and P9 positions in I₂₁₆VYNYANAS. The near background mean fluorescence of the initial library cell population (10⁷ transformants) revealed that the overwhelming majority of amino acid substitutions at these residues are deleterious. Nonetheless, a population with near WT fluorescence was established after 4 rounds of FACS sorting (FIG. 5B). In contrast to the high degree of plasticity observed in the M₁₁₅ core region, mutagenesis of the MHC anchor positions in the I₂₁₆ core yielded mostly conservative amino acid substitutions (FIG. 2A). One variant of 1.1.C4 containing two mutations, a conservative change at 1216V and a non-conservative one a N219G, displayed a 7-fold increase in CPR score of the modified I₂₁₆ core region for DRB1*0401, and increases in the CPR scores for 7 other common MHC-II alleles (Table 1). Once again, these mutations did not affect the catalytic properties of the enzyme for the hydrolysis of L-Asn (k_{cat}=21 s⁻¹, K_{M}=19 μM, k_{cat}/K_{M}=1.1×10⁶ M⁻¹s⁻¹). This variant, designated 2.2.G10, was then used as a template for mutagenesis of the V₃₀₄LLQLALTQ T-cell epitope core region (10⁷ transformants). The final enzyme variant, designated 3.1.E2, further containing a non-conservative change at Q307T and a conservative Q312N substitution, showed a 3-fold increase in the CPR score for binding to DRB1*0401 and increased CPR scores for 4 other alleles (Table 1). 3.1.E2 contained a total of 8 amino acid substitutions (FIG. 2B), but retained a k_{cat} (L-Asn) identical to the parent enzyme with just a 3-fold increase in K_{M} (L-Asn) (k_{cat}=24 s⁻¹, K_{M}=23 μM, k_{cat}/K_{M}=1.0×10⁶ M⁻¹s⁻¹). 3.1.E2 further displayed slightly reduced (33%) specific activity towards L-Gln hydrolysis that may be of therapeutic benefit (42), was stable in serum for over 10 days (FIG. 6); essentially identical to the WT EcAII (46), and could be expressed at a high yield (>30 mg/L shake flask culture) (FIG. 7). Finally, the inventorsnote that because recombinant EcAII folds and expresses well, no mutants with low activity but compensatory expression levels were isolated in screening each library.

### Example 5

### Evaluation of EcAII T-Cell Responses and Immunogenicity Using HLA-Transgenic Mice

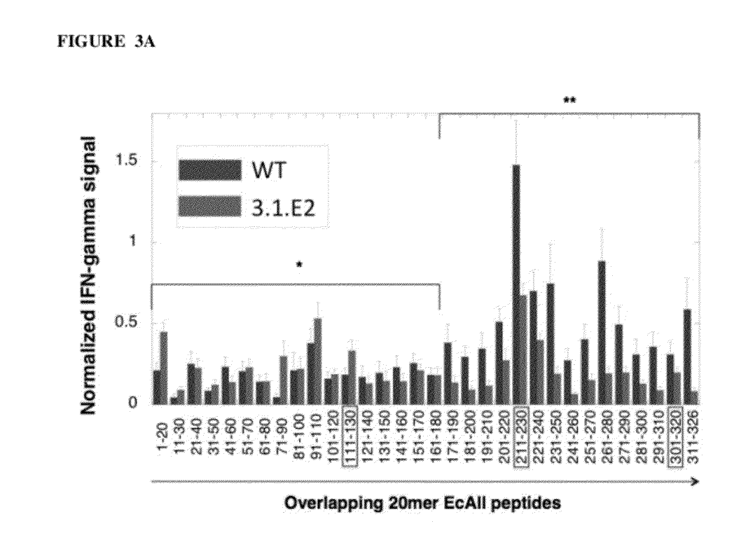

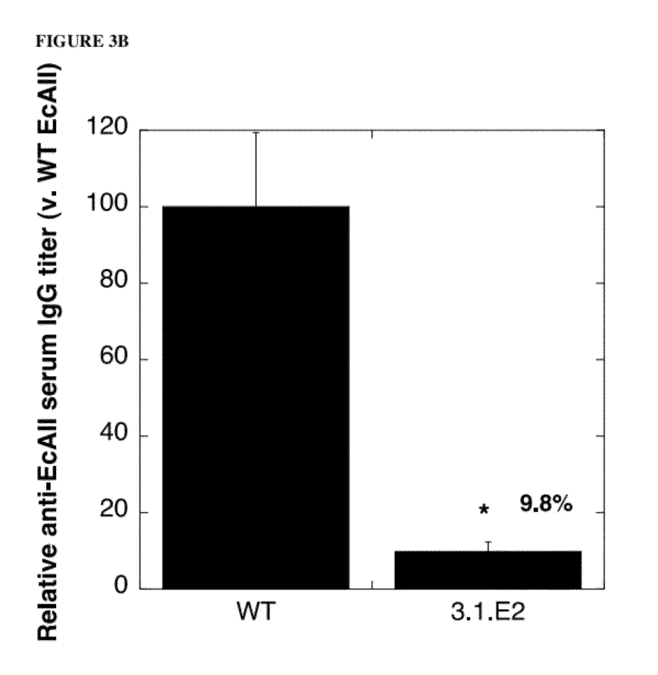

The immunogenicity of purified, low endotoxin preparations of WT EcAII and 3.1.E2 were evaluated in transgenic mice expressing human HLA-DRB1*0401 under the mouse MHC-II promoter and deficient in the endogenous murine MHC-II locus. As a stringent test of the potential for immunogenicity, mice were immunized with a strong adjuvant (Complete Freund's Adjuvant) to induce robust CD4+ T-cell responses. The HLA transgenic mice were immunized with either WT EcAII or 3.1.E2 and T-cell responses were measured in draining lymph node cells by cytokine ELISPOT assays for IFN-γ levels following recall with either the initial enzyme itself or with overlapping 20-mer synthetic peptides corresponding to the sequence of the enzyme used in the initial immunization (FIG. 3A). For WT EcAII, the highest level of T-cell activation was observed in response to 20-mer WTp211-230, which contained the predicted core region I₂₁₆VYNYANAS. Deimmunization resulted in a significant decrease in T-cell activation by peptides containing the mutated sequences relative to I₂₁₆VYNYANAS and V₃₀₄LLQLALTYQ in the parental enzyme. In contrast, although mutagenesis of the M₁₁₅RPSTSMSA region resulted in a sequence with improved CPR score with for DRB1*0401, no statistical difference in cytokine stimulation could be observed for the mutant peptide. This was probably a consequence of the complex relationship between antigen processing, MHC-II binding, and TCR recognition and signaling (47). Interestingly, while the cytokine responses induced by the 17 N-terminal overlapping peptides were essentially indistinguishable (p=0.182) regardless of whether the mice had been immunized with WT or mutant enzyme, a significantly reduced response (p<0.0001) was observed for the 3.1.E2-immunized population across the 15 C-terminal overlapping peptides. One possible explanation for this result is that the mutations in 3.1.E2 may have affected antigen processing, thus altering MHC-II loading (48). Importantly, mice immunized with 3.1.E2 also displayed a statistically significant (p=0.02) 10-fold reduction in anti-EcAII IgG titer relative to mice receiving the WT enzyme (FIG. 3B). Given that the activation of CD4+ T-cells is in most cases required for the longevity and proliferation of B-cells and for antibody isotype-switching (49), this result strongly implicated that the removal of EcAII T-cell epitopes in 3.1.E2 resulted in reduced T-cell help and thus led to lower antibody titers relative to the WT enzyme.

### Example 6

This example decribes the development of a screen according to the methods taught by Example 2 for the enrichment of cells expressing methioninase enzyme. Specifically in this example cells expressing the P. putida methionine-gamma-lyase enzyme (pMGL) were enriched from a very large excess of cells displaying a structurally homologous enzyme not displaying methionine gamma lyase activity by FACS.

E. coli BL21(DE3)(ΔilvA, ΔmetA) a strain auxotrophic for L-isoleucine and L-methionine can be rescued if supplemented with L-Met while harboring a plasmid containing the gene for a methionine-γ-lyase, resulting in complementation of the ilvA deletion by production of alpha-ketobutyrate. The metA deletion prevents formation of cystathionine and ensures that cell containing genes with cystathionine-y-lyase activity will not rescue the L-isoleucine auxotrophy.

E. coli BL21(DE3)(ΔilvA, ΔmetA) cells carrying an IPTG-inducible pET28a plasmid containing the gene for the P. putida methionine-gamma-lyase enzyme (pMGL) gene were spiked at a ratio of 1 in 10,000 into a pool of cells carrying the pET28a plasmid containing the gene for the human cystathionine-gamma-lyase (hCGL). Additionally, all cells were transformed with the pBAD-GFP reporter plasmid. The cell mixture was grown in M9 medium supplemented with 0.4% glucose, 1 mM MgSO₄, 0.1 mM CaCl₂, 140 mg/L of L-isoleucine and L-methionine, 70 mg/L of all remaining amino acids excluding L-leucine and L-valine, 50 μg/ml kanamycin, and 34 μg/ml chloramphenicol. The cultures were incubated at 37° C. until they reached an OD₆₀₀=0.3-0.4, at which point enzyme expression was induced by the addition of 1 mM IPTG. The cultures were shifted to 25° C. for three hours, at which point they were harvested by centrifugation (7,000×g, 3 min), washed twice with cold 0.9% NaCl, and resuspended in supplemented M9 medium with the following modifications: no L-isoleucine, 50 μM IPTG, and 2% arabinose for the induction of GFP expression. After two hours of expression at 25° C., the cells were diluted in PBS to a final OD₆₀₀˜0.05 for flow cytometric analysis and cell sorting.

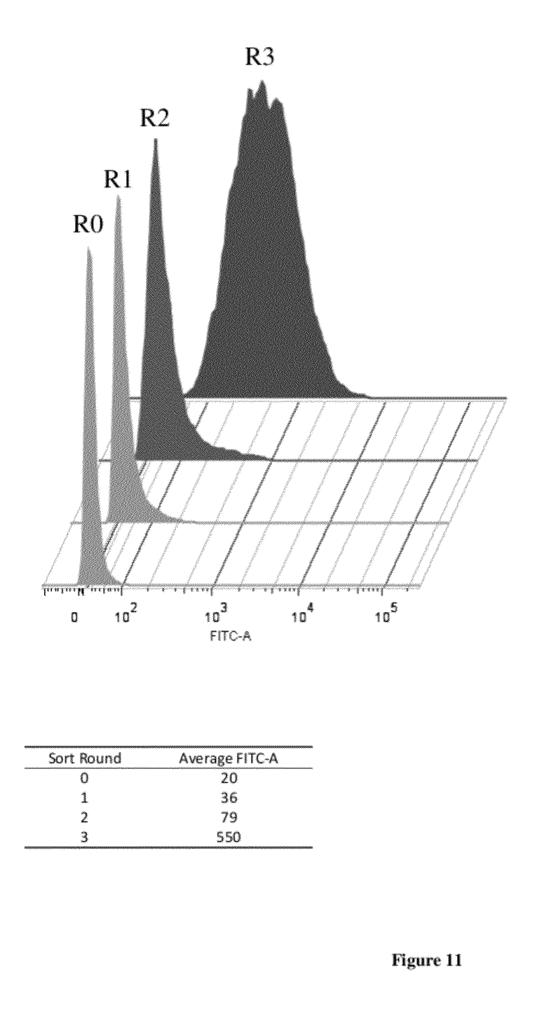

Flow cytometric analyses were performed with a FACSAria (BD Biosciences) using a 488-nm solid-state laser for excitation and a 530/30 band pass filter for detection. The throughput rate of cells was adjusted to 4,000-5,000 events per second, and ˜10⁵ cells were sorted each round in single cell mode except for the initial sort of the population, which was done in purity mode. A gate in the fluorescence channel was set to recover the 4-5% most highly fluorescent cells while additional gates were set based on both the forward- and side-scatter channels to exclude sorting nonsingle cell events. The sorted cells were collected in 0.5 mL of SOB medium and then plated onto 2×YT medium agar supplemented with 50 μg/mL kanamycin and 34 μg/mL chloramphenicol. After overnight growth at 30° C., the colonies were scraped, pooled and used as the source for inoculation for the following round. A total of three rounds of sorting brought the average FITC-A signal from the negative control baseline fluorescence (−20) to that of the positive control (−600) (FIG. 11). A total of 12 clones from the third round of sorting were submitted for sequencing, 50% of which were determined to correspond to the sequence of pMGL. Therefore, three rounds were sufficient to achieve a 5,000 fold enrichment of the positive control-expressing clones (FIG. 4).

### REFERENCES CITED IN THE SPECIFICATION AND EXAMPLES 1-5

1. Leader B, Baca Q J, & Golan D E (2008) Protein therapeutics: a summary and pharmacological classification. Nat Rev Drug Discov 7(1):21-39.
2. Kim W, et al. (2004) Trends in enzyme therapy for phenylketonuria. Mol Ther 10(2):220-224.
3. Chohan S & Becker M A (2009) Update on emerging urate-lowering therapies. Curr Opin Rheumatol 21(2):143-149.
4. Ni Y, Schwaneberg U, & Sun Z H (2008) Arginine deiminase, a potential anti-tumor drug. Cancer Lett 261(1):1-11.
5. Tan Y, Xu M, & Hoffman R M (Broad selective efficacy of recombinant methioninase and polyethylene glycol-modified recombinant methioninase on cancer cells In Vitro. Anticancer Res 30(4):1041-1046.
6. Cooney D A & Handschumacher R E (1970) L-asparaginase and L-asparagine metabolism. Annu Rev Pharmacol 10:421-440.
7. Haskell C M, Canellos G P, Cooney D A, & Hansen H H (1970) Biocmical and pharmacologic effects of L-asparaginase in man. J Lab Clin Med 75(5):763-770.
8. Rytting M (2010) Peg-asparaginase for acute lymphoblastic leukemia. Expert Opin Biol Ther 10(5):833-839.
9. Schellekens H (2002) Immunogenicity of therapeutic proteins: Clinical implications and future prospects. Clin Ther 24(11):1720-1740.
10. Jevsevar S, Kunstelj M, & Porekar V G (2010) PEGylation of therapeutic proteins. Biotechnol J 5(1):113-128.
11. Armstrong J K, et al. (2007) Antibody against poly(ethylene glycol) adversely affects PEG-asparaginase therapy in acute lymphoblastic leukemia patients. Cancer 110(1): 103-111.
12. Sherman M R, Saifer M G P, & Perez-Ruiz F (2008) PEG-uricase in the management Of treatment-resistant gout and hyperuricemia. Adv Drug Deliv Rev 60(1):59-68.
13. Yeung V P, et al. (2004) Elimination of an immunodominant CD4+ T cell epitope in human IFN-beta does not result in an in vivo response directed at the subdominant epitope. J Immunol 172(11):6658-6665.
14. Harding F A, et al. (2005) A beta-lactamase with reduced immunogenicity for the targeted delivery of chemotherapeutics using antibody-directed enzyme prodrug therapy. Mol Cancer Ther 4(11):1791-1800.
15. Jones E Y, Fugger L, Strominger J L, & Siebold C (2006) MHC class II proteins and disease: a structural perspective. Nat Rev Immunol 6(4): 271-282.
16. Wang P, et al. (2008) A systematic assessment of MHC class II peptide binding predictions and evaluation of a consensus approach. PLoS Comput Biol 4(4): e1000048.
17. Bander N H, et al. (2005) Phase I trial of 177lutetium-labeled J591, a monoclonal antibody to prostate-specific membrane antigen, in patients with androgen-independent prostate cancer. J Clin Oncol 23(21):4591-4601.
18. Macfarlane D J, et al. (2006) Safety, pharmacokinetic and dosimetry evaluation of the proposed thrombus imaging agent 99 mTc-DI-DD-3B6/22-80B3 Fab′. Eur J Nucl Med Mol Imaging 33(6):648-656.
19. Holgate R G & Baker M P (2009) Circumventing immunogenicity in the development of therapeutic antibodies. IDrugs 12(4):233-237.
20. Benkovic S J & Hammes-Schiffer S (2003) A perspective on enzyme catalysis. Science 301(5637):1196-1202.
21. Warmerdam P A M, et al. (2002) Elimination of a human T-cell region in staphylokinase by T-cell screening and computer modeling. Thromb Haemost 87(4):666-673.
22. Amitai G, Gupta R D, & Tawfik D S (2007) Latent evolutionary potentials under the neutral mutational drift of an enzyme. HFSP J 1(1):67-78.
23. Bloom J D, Romero P A, Lu Z, & Arnold F H (2007) Neutral genetic drift can alter promiscuous protein functions, potentially aiding functional evolution. Biol Direct 2:17.
24. Bershtein S & Tawfik D S (2008) Advances in laboratory evolution of enzymes. Curr Opin Chem Biol 12(2):151-158.
25. Gupta R D & Tawfik D S (2008) Directed enzyme evolution via small and effective neutral drift libraries. Nat Methods 5(11):939-942.
26. Link A J, Jeong K J, & Georgiou G (2007) Beyond toothpicks: new methods for isolating mutant bacteria. Nat Rev Microbiol 5(9):680-688.
27. Breaker R R, Banerji A, & Joyce G F (1994) Continuous in-Vitro Evolution of Bacteriophage Rna-Polymerase Promoters. Biochemistry 33(39):11980-11986.
28. Gates C M, Stemmer W P, Kaptein R, & Schatz P J (1996) Affinity selective isolation of ligands from peptide libraries through display on a lac repressor “headpiece dimer”. J Mol Biol 255(3):373-386.
29. Yano T & Kagamiyama H (2001) Directed evolution of ampicillin-resistant activity from a functionally unrelated DNA fragment: A laboratory model of molecular evolution. Proc Natl Acad of Sci USA 98(3):903-907.
30. Broome J D (1963) Evidence that the L-asparaginase of guinea pig serum is responsible for its antilymphoma effects. II. Lymphoma 6C3HED cells cultured in a medium devoid of L-asparagine lose their susceptibility to the effects of guinea pig serum in vivo. J Exp Med 118:121-148.
31. Asselin B L, et al. (1989) In vitro and in vivo killing of acute lymphoblastic leukemia cells by L-asparaginase. Cancer Res 49(15):4363-4368.
32. Avramis V I, et al. (2002) A randomized comparison of native Escherichia coli asparaginase and polyethylene glycol conjugated asparaginase for treatment of children with newly diagnosed standard-risk acute lymphoblastic leukemia: a Children's Cancer Group study. Blood 99(6):1986-1994.
33. Pui C-H & Evans W E (2006) Treatment of acute lymphoblastic leukemia. N Engl J Med 354(2):166-178.
34. Horowitz B, et al. (1968) Asparagine synthetase activity of mouse leukemias. Science 160(827):533-535.
35. Ho D H, Whitecar J P, Jr., Luce J K, & Frei E, 3rd (1970) L-asparagine requirement and the effect of L-asparaginase on the normal and leukemic human bone marrow. Cancer Res 30(2):466-472.
36. Capizzi R L, et al. (1971) L-asparaginase: clinical, biochemical, pharmacological, and immunological studies. Ann Intern Med 74(6):893-901.
37. Story M D, Voehringer D W, Stephens L C, & Meyn R E (1993) L-asparaginase kills lymphoma cells by apoptosis. Cancer Chemother Pharmacol 32(2):129-133.
38. Ueno T, et al. (1997) Cell cycle arrest and apoptosis of leukemia cells induced by L-asparaginase. Leukemia 11(11):1858-1861.
39. Zeidan A, Wang E S, & Wetzler M (2009) Pegasparaginase: where do we stand? Expert Opin Biol Ther 9(1):111-119.
40. Avramis V I & Tiwari P N (2006) Asparaginase (native ASNase or pegylated ASNase) in the treatment of acute lymphoblastic leukemia. Int Journal Nanomedicine 1(3): 241-254.
41. Duval M, et al. (2002) Comparison of Escherichia coli-asparaginase with Erwinia-asparaginase in the treatment of childhood lymphoid malignancies: results of a randomized European Organisation for Research and Treatment of Cancer-Children's Leukemia Group phase 3 trial. Blood 99(8): 2734-2739.
42. Derst C, Henseling J, & Röhm K H (2000) Engineering the substrate specificity of Escherichia coli asparaginase. II. Selective reduction of glutaminase activity by amino acid replacements at position 248. Protein Sci 9(10):2009-2017.
43. Asselin B L, et al. (1991) Measurement of serum L-asparagine in the presence of L-asparaginase requires the presence of an L-asparaginase inhibitor. Cancer Res 51(24):6568-6573.
44. Dorak M T, et al. (1999) Unravelling an HLA-DR association in childhood acute lymphoblastic leukemia. Blood 94(2): 694-700.
45. Southwood S, et al. (1998) Several common HLA-DR types share largely overlapping peptide binding repertoires. J Immunol 160(7): 3363-3373.
46. Stecher A L, de Deus P M, Polikarpov I, & Abrahalo-Neto J (1999) Stability of L-asparaginase: an enzyme used in leukemia treatment. Pharm Acta Hely 74(1): 1-9.
47. Huang J, et al. (2010) The kinetics of two-dimensional TCR and pMHC interactions determine T-cell responsiveness. Nature 464(7290): 932-936.
48. Jensen P E (2007) Recent advances in antigen processing and presentation. Nat Immunol 8(10): 1041-1048.
49. McHeyzer-Williams U & McHeyzer-Williams M G (2005) Antigen-specific memory B cell development. Annu Rev Immunol 23:487-513.
50. Stoop A A & Craik C S (2003) Engineering of a macromolecular scaffold to develop specific protease inhibitors. Nat Biotechnol 21(9):1063-1068.
51. Chen J C, Viollier P H, & Shapiro L (2005) A membrane metalloprotease participates in the sequential degradation of a Caulobacter polarity determinant. Mol Microbiol 55(4):1085-1103.
52. Link A J, Skretas G, Strauch E M, Chari NS, & Georgiou G (2008) Efficient production of membrane-integrated and detergent-soluble G protein-coupled receptors in Escherichia coli. Protein Sci 17(10):1857-1863.
53. Yoo T H, Link A J, & Tirrell D A (2007) Evolution of a fluorinated green fluorescent protein. Proc Natl Acad Sci USA 104(35):13887-13890.
54. Ito K, et al. (1996) HLA-DR4-IE chimeric class II transgenic, murine class II-deficient mice are susceptible to experimental allergic encephalomyelitis. J Exp Med 183(6):2635-2644.
55. DeLano D W (2002) The PyMol Graphics System. DeLano Scientific, Palo Alto, Calif.

### ADDITIONAL REFERENCES CITED IN EXAMPLE 1

1. Derst C, Henseling J, & Röhm K H (2000) Engineering the substrate specificity of Escherichia coli asparaginase. II. Selective reduction of glutaminase activity by amino acid replacements at position 248. Protein Sci 9(10):2009-2017.
2. Sanches M, Krauchenco S, & Polikarpov I (2007) Structure, Substrate Complexation and Reaction Mechanism of Bacterial Asparaginases. Curr Chem Biol 1(1):75-86.
3. Skerra A (1994) Use of the tetracycline promoter for the tightly regulated production of a murine antibody fragment in Escherichia coli. Gene 151(1-2):131-135.
4. Yoo T H, Link A J, & Tirrell D A (2007) Evolution of a fluorinated green fluorescent protein. Proc Natl Acad Sci USA 104(35):13887-13890.
5. Datsenko K A & Wanner B L (2000) One-step inactivation of chromosomal genes in Escherichia coli K-12 using PCR products. Proc Natl Acad Sci USA 97(12):6640-6645.
6. Sturniolo T, et al. (1999) Generation of tissue-specific and promiscuous HLA ligand databases using DNA microarrays and virtual HLA class II matrices. Nat Biotechnol
17(6):555-561.
7. Singh H & Raghava G P (2001) ProPred: prediction of HLA-DR binding sites. Bioinformatics 17(12):1236-1237.
8. Wang P, et al. (2008) A systematic assessment of MHC class II peptide binding predictions and evaluation of a consensus approach. PLoS Comput Biol 4(4): e1000048.
9. Southwood S, et al. (1998) Several common HLA-DR types share largely overlapping peptide binding repertoires. in J Immunol 160(7): 3363-3373.
10. Wehner A, et al. (1992) Site-specific mutagenesis of Escherichia coli asparaginase II. None of the three histidine residues is required for catalysis. Eur J Biochem 208(2): 475-480.
11. Cantor J R, Stone E M, Chantranupong L, & Georgiou G (2009) The human asparaginase-like protein 1 hASRGL1 is an Ntn hydrolase with beta-aspartyl peptidase activity. Biochemistry 48(46):11026-11031.
12. Aida Y & Pabst M J (1990) Removal of endotoxin from protein solutions by phase separation using Triton X-114. J Immunol Methods 132(2):191-195.
13. Forsthuber T G, et al. (2001) T cell epitopes of human myelin oligodendrocyte glycoprotein identified in HLA-DR4 (DRB1*0401) transgenic mice are encephalitogenic and are presented by human B cells. J Immunol 167(12):7119-7125.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiment, it should be understood that the invention as claimed should not be unduly limited to such specific embodiments. Indeed, various modifications of the described modes for carrying out the invention which are obvious to those skilled in the art and in fields related thereto are intended to be within the scope of the following claims.

We claim:

1. A mutant L-asparaginase that
A) comprises 8 amino acid substitutions that correspond to substitution of wild type L-asparaginase SEQ ID NO:03
1) methionine at position 115 with valine (M115V),
2) serine at position 118 with proline (S118P),
3) serine at position 120 with arginine (S120R),
4) alanine at position 123 with proline (A123P),
5) isoleucine at position 215 with valine (1215V),
6) asparagine at position 219 with glycine (N219G),
7) glutamine at position 307 with threonine (Q307T), and
8) glutamine at position 312 with asparagine (Q312N)
B) has the same or greater enzyme activity as wild type L-asparaginase SEQ ID NO:03, and
C) has reduced immunogenicity compared to wild type L-asparaginase SEQ ID NO:03.

2. The mutant L-asparaginase of claim 1, wherein said mutant L-asparaginase has the same or greater stability of enzyme activity in serum as wild type L-asparaginase SEQ ID NO:03.

3. The mutant L-asparaginase of claim 1, wherein said mutant L-asparaginase comprises SEQ ID NO:01.

4. A pharmaceutical composition comprising the mutant L-asparaginase of claim 1, and a carrier.

5. A recombinant nucleotide sequence encoding the mutant L-asparaginase of claim 1.

6. The recombinant nucleotide sequence of claim 5, wherein said nucleotide sequence comprises SEQ ID NO:02.

7. An expression vector that comprises a nucleotide sequence encoding the mutant L-asparaginase of claim 1.

8. A transgenic cell comprising the expression vector of claim 7.

9. A method for identifying a mutant deimmunized protein that has the same or greater biological activity as a protein of interest, comprising
A) providing
i) a first plurality of first expression vectors, wherein each expression vector comprises in operable combination
1) a first nucleotide sequence encoding a mutant of a protein of interest, wherein said protein of interest comprises one or more epitope sequence, and wherein said mutant protein contains one or more mutations in one or more of said epitope sequence,
2) a reporter nucleotide sequence, and
3) a promoter,
ii) a transgenic cell that lacks expression of a biologically active said protein of interest,
B) transfecting said transgenic cell with said first plurality of first expression vectors to produce a first plurality of populations of transfected transgenic cells, wherein each population of said first plurality of populations of transfected transgenic cells comprises one of said first expression vectors,
C) culturing said first plurality of populations of transfected transgenic cells under conditions for expression of said first nucleotide sequence and of said reporter nucleotide sequence,
D) detecting expression of said reporter nucleotide sequence in said first plurality of populations of transfected transgenic cells, wherein the level of said expression of said reporter nucleotide sequence correlates with the level of biological activity of said mutant protein that is encoded by said operably linked first nucleotide sequence, and
E) determining the immunogenicity in said first plurality of populations of transfected transgenic cells of said expressed mutant protein,
wherein
i) detecting the same or greater biological activity of said mutant protein compared to said protein of interest, and
ii) detecting reduced immunogenicity of said mutant protein compared to said protein of interest,
identifies said mutant protein as a deimmunized protein that has the same or greater biological activity as said protein of interest.

10. The method of claim 9, further comprising
F) providing
i) a second plurality of second expression vectors, wherein each expression vector of said second plurality of expression vectors comprises in operable combination
1) a second nucleotide sequence encoding a variant of said identified mutant protein, wherein said variant protein contains additional one or more mutations in said one or more epitope sequence of said identified mutant protein,
2) a reporter nucleotide sequence, and
3) a promoter,
ii) a transgenic cell that lacks expression of a biologically active said protein of interest,
G) transfecting said transgenic cell with said second plurality of expression vectors to produce a second plurality of populations of transfected transgenic cells, wherein each population of said second plurality of populations of transfected transgenic cells comprises one of said second expression vectors,
H) culturing said second plurality of populations of transfected transgenic cells under conditions for expression of said second nucleotide sequence and said reporter nucleotide sequence,
I) detecting expression of said reporter nucleotide sequence in said second plurality of populations of transfected transgenic cells, wherein the level of said expression of said reporter nucleotide sequence correlates with the level of biological activity of said variant protein that is encoded by said operably linked second nucleotide sequence, and
J) determining the immunogenicity in said plurality of populations of transfected transgenic cells of said expressed variant protein,
wherein
i) detecting the same or greater biological activity of said variant protein compared to said protein of interest, and
ii) detecting reduced immunogenicity of said variant protein compared to said protein of interest,
identifies said variant protein as a deimmunized protein that has the same or greater biological activity as said protein of interest.

11. The method of claim 10, further comprising detecting the stability of said biological activity of said mutant protein.

12. The method of claim 9, further comprising purifying the identified mutant deimmunized protein.

13. The method of claim 12, further comprising detecting one or more mutation in the epitope sequence of said purified mutant deimmunized protein.

14. The method of claim 12, further comprising determining immunogenicity of said purified mutant deimmunized protein.

15. The method of claim 9, wherein said protein of interest is an enzyme, and said transgenic cell further lacks expression of a product produced by the enzyme activity of a wild type of said enzyme.

16. The method of claim 9, wherein said reporter nucleotide sequence comprises a gene encoding a fluorescent protein.

17. The method of claim 9, wherein said protein of interest is selected from the group consisting of enzyme of interest and binding protein of interest.

18. The method of claim 17, wherein said enzyme of interest is an amino acid degrading enzyme.

19. The method of claim 18, wherein said amino acid degrading enzyme comprises L-Asparaginase.

20. The method of claim 9, wherein said reduced immunogenicity comprises from 1 to 10,000 fold lower immunogenicity of said mutant protein compared to immunogenicity of said protein of interest.

21. A pharmaceutical composition comprising the mutant protein identified by the method of claim 9, and a carrier.

22. A method for reducing immunogenicity of a protein of interest without reducing biological activity of said protein of interest, comprising
a). identifying a mutant of said protein of interest using the method of claim 9,
b) determining the amino acid sequence of one or more said epitope sequence in said identified mutant protein, and
c) producing a variant protein of interest that contains the determined epitope sequence.

23. A pharmaceutical composition comprising the variant protein of interest produced by the method of claim 22, and a carrier.

24. A method for treating disease comprising administering to a mammalian subject in need thereof a therapeutically effective amount of a pharmaceutical composition comprising at least one protein selected from the group consisting of
a) the mutant L-asparaginase of claim 1,
b) the mutant deimmunized protein identified by the method of claim 9, and
c) the variant protein of interest produced by the method of claim 22.

25. The method of claim 24, wherein said protein is heterologous to said subject.

26. The method of claim 24, wherein said protein is selected from the group consisting of enzyme and binding protein.

27. A method for identifying a mutant mammalian enzyme that has a desired level of catalytic activity for degradation of an amino acid, comprising:
A) providing
i) a plurality of expression vectors, wherein each expression vector comprises in operable combination
1) a nucleotide sequence encoding a mutant of said mammalian enzyme,
2) a reporter nucleotide sequence, and
3) a promoter, and
ii) a transgenic cell that lacks expression of enzymes having said catalytic activity,
B) transfecting said transgenic cell with said plurality of expression vectors to produce a plurality of populations of transfected transgenic cells, wherein each population of transfected transgenic cells comprises one of said expression vectors,
C) culturing said plurality of populations of transfected transgenic cells under conditions for expression of the nucleotide sequence encoding said mutant and for expression of the reporter nucleotide sequence, and
D) detecting expression of the reporter nucleotide sequence in one or more of the plurality of populations of transfected transgenic cells, wherein the level of expression of said reporter nucleotide sequence correlates with the level of said catalytic activity of said mammalian mutant enzyme that is encoded by said operably linked nucleotide sequence, and wherein said detecting identifies said mutant mammalian enzyme as having a desired level of said catalytic activity.


SEQUENCE LISTING (79 sequences)

SEQ ID NO: 1 - protein, 326 aa, Escherichia coli
LPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 2 - DNA, 978 bp, Escherichia coli
ttacccaatatcaccattttagcaaccggcgggaccattgccggtggtggtgactccgcaaccaaatctaactacacagtgggtaaagttggcgtagaaaatctggttaatgcggtgccgcaactaaaagacattgcgaacgttaaaggcgagcaggtagtgaatatcggctcccaggacatgaacgataatgtctggctgacactggcgaaaaaaattaacaccgactgcgataagaccgacggcttcgtcattacccacggtaccgacacgatggaagaaactgcttacttcctcgacctgacggtgaaatgcgacaaaccggtggtgatggtcggcgcaatgcgtccgtccacgtctatgagcgcagacggtccattcaacctgtataacgcggtagtgaccgcagctgataaagcctccgccaaccgtggcgtgctggtagtgatgaatgacaccgtgcttgatggccgtgacgtcaccaaaaccaacaccaccgacgtagcgaccttcaagtctgttaactacggtcctctgggttacattcacaacggtaagattgactaccagcgtaccccggcacgtaagcataccagcgacacgccattcgatgtctctaagctgaatgaactgccgaaagtcggcattgtttataactacgctaacgcatccgatcttccggctaaagcactggtagatgcgggctatgatggcatcgttagcgctggtgtgggtaacggcaacctgtataaatctgtgttcgacacgctggcgaccgccgcgaaaaccggtactgcagtcgtgcgttcttcccgcgtaccgacgggcgctaccactcaggatgccgaagtggatgatgcgaaatacggcttcgtcgcctctggcacgctgaacccgcaaaaagcgcgcgttctgctgcaactggctctgacgcaaaccaaagatccgcagcagatccagcagatcttcaatcagtac

SEQ ID NO: 3 - protein, 326 aa, Escherichia coli
LPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAVRPPTRMSPDGPFNLYNAVVTAADKASANRGVLVVMNDTVLDGRDVTKTNTTDVATFKSVNYGPLGYIHNGKIDYQRTPARKHTSDTPFDVSKLNELPKVGVVYGYANASDLPAKALVDAGYDGIVSAGVGNGNLYKSVFDTLATAAKTGTAVVRSSRVPTGATTQDAEVDDAKYGFVASGTLNPQKARVLLTLALTNTKDPQQIQQIFNQY

SEQ ID NO: 4 - DNA, 978 bp, Escherichia coli
ttacccaatatcaccattttagcaaccggcgggaccattgccggtggtggtgactccgcaaccaaatctaactacacagtgggtaaagttggcgtagaaaatctggttaatgcggtgccgcaactaaaagacattgcgaacgttaaaggcgagcaggtagtgaatatcggctcccaggacatgaacgataatgtctggctgacactggcgaaaaaaattaacaccgactgcgataagaccgacggcttcgtcattacccacggtaccgacacgatggaagaaactgcttacttcctcgacctgacggtgaaatgcgacaaaccggtggtgatggtcggcgcagtccgtccgcccacgcgcatgagccccgacggtccattcaacctgtataacgcggtagtgaccgcagctgataaagcctccgccaaccgtggcgtgctggtagtgatgaatgacaccgtgcttgatggccgtgacgtcaccaaaaccaacaccaccgacgtagcgaccttcaagtctgttaactacggtcctctgggttacattcacaacggtaagattgactaccagcgtaccccggcacgtaagcataccagcgacacgccattcgatgtctctaagctgaatgaactgccgaaagtcggcgtggtttatgggtacgcgaacgcaagcgatcttccggctaaagcactggtagatgcgggctatgatggcatcgttagcgctggtgtgggtaacggcaacctgtataaatctgtgttcgacacgctggcgaccgccgcgaaaaccggtactgcagtcgtgcgttcttcccgcgtaccgacgggcgctaccactcaggatgccgaagtggatgatgcgaaatacggcttcgtcgcctctggcacgctgaacccgcaaaaagcgcgcgtcctgctgaccctggccctgacgaacaccaaagatccgcagcagatccagcagatcttcaatcagtac

SEQ ID NO: 5 - protein, 410 aa, Mycoplasma arginini
MSVFDSKFKGIHVYSEIGELESVLVHEPGREIDYITPARLDELLFSAILESHDARKEHKQFVAELKANDINVVELIDLVAETYDLASQEAKDKLIEEFLEDSEPVLSEEHKVVVRNFLKAKKTSRELVEIMMAGITKYDLGIEADHELIVDPMPNLYFTRDPFASVGNGVTIHYMRYKVRQRETLFSRFVFSNHPKLINTPWYYDPSLKLSIEGGDVFIYNNDTLVVGVSERTDLQTVTLLAKNIVANKECEFKRIVAINVPKWTNLMHLDTWLTMLDKDKFLYSPIANDVFKFWDYDLVNGGAEPQPVENGLPLEGLLQSIINKKPVLIPIAGEGASQMEIERETHFDGTNYLAIRPGVVIGYSRNEKTNAALEAAGIKVLPFHGNQLSLGMGNARCMSMPLSRKDVKW

SEQ ID NO: 6 - protein, 398 aa, Mycoplasma arginini
MHGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 7 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 8 - protein, 302 aa, Aspergillus flavus
MSAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 9 - protein, 162 aa, Escherichia coli
MKTILPAVLFAAFATTSAWAAESVQPLEKIAPYPQAEKGMKRQVIQLTPQEDESTLKVELLIGQTLEVDCNLHRLGGKLENKTLEGWGYDYYVFDKVSSPVSTMMACPDGKKEKKFVTAYLGDAGMLRYNSKLPIVVYTPDNVDVKYRVWKAEEKIDNAVVR

SEQ ID NO: 10 - protein, 335 aa, Burkholderia xenovorans
MLKEALASAETVAAQLMDTSRVEALAAKLAQQPRHVALTVARGSSDHAASYFASLTMSRLGVPVASLPMSVATLQQAPLQVCDQLALAFSQSGKSPDLVGTMEALRKAGALTVAAVNVSGSPLADACEFELPLVAGPELSVAATKSYIAMLSISAQLVAHWQKDAELLAALKTLPEALRAAGKLDWSNAVEELRGVERMIVIGRGLGLAIAQEAALKLKETSGIQAEAFSSAEVRHGPMELIDRDYPLLVFAPRGPEQAGLLQLARDMQARGARVLLAAPADVPEATLPLAITAHAALDPIAAILSFYVMAAGLAAARGRNPDAPRHLNKVTETH

SEQ ID NO: 11 - DNA, 30 bp, Artificial Sequence
  Synthetic
gtgcagcacatatgttacccaatatcacca

SEQ ID NO: 12 - DNA, 27 bp, Artificial Sequence
  Synthetic
ggcgggatccttagtactgattgaaga

SEQ ID NO: 13 - DNA, 51 bp, Artificial Sequence
  Synthetic
gtgcagcacatatgttacccaatatcaccattttagcaaccggcggggcca

SEQ ID NO: 14 - DNA, 19 bp, Artificial Sequence
  Synthetic
gctagttattgctcagcgg

SEQ ID NO: 15 - DNA, 35 bp, Artificial Sequence
  Synthetic
ctagctagtctagagcggagcctatggaaaaacgc

SEQ ID NO: 16 - DNA, 34 bp, Artificial Sequence
  Synthetic
catgccatggcacttttctctatcactgataggg

SEQ ID NO: 17 - DNA, 52 bp, Artificial Sequence
  Synthetic
gggccggcaggcgctccattgcccagtcggctaagggattttggtcatgaac

SEQ ID NO: 18 - DNA, 48 bp, Artificial Sequence
  Synthetic
gcgtagcgaccgagtgagctagcttattagaaaaactcatcgagcatc

SEQ ID NO: 19 - DNA, 70 bp, Artificial Sequence
  Synthetic
cctcacgtatatacttttgctctttcgatatcattcatatcaatatcatgattccggggatccgtcgacc

SEQ ID NO: 20 - DNA, 70 bp, Artificial Sequence
  Synthetic
acagggcgcgagggggcattacagtctccttaatcatccggcgtcagttctgtaggctggagctgcttcg

SEQ ID NO: 21 - DNA, 70 bp, Artificial Sequence
  Synthetic
cagagctaagggataatgcgtagcgttcacgtaactggaggaatgaaatgattccggggatccgtcgacc

SEQ ID NO: 22 - DNA, 70 bp, Artificial Sequence
  Synthetic
agccccggcacgataccggggcgaggcgattagtactgattgaagatctgtgtaggctggagctgcttcg

SEQ ID NO: 23 - DNA, 70 bp, Artificial Sequence
  Synthetic
tgatatttatagcaaaagtggcgaaccacccttaatggacgaatactatgattccggggatccgtcgacc

SEQ ID NO: 24 - DNA, 70 bp, Artificial Sequence
  Synthetic
ccgccagcacattaccggcatcaagttcatcactgtgtggcaacggtgtctgtaggctggagctgcttcg

SEQ ID NO: 25 - DNA, 70 bp, Artificial Sequence
  Synthetic
taccctgatagcggacttcccttctgtaaccataatggaacctcgtcatgattccggggatccgtcgacc

SEQ ID NO: 26 - DNA, 70 bp, Artificial Sequence
  Synthetic
ttttcagcgggcttcattgtttttaatgcttacagcactgccacaatcgctgtaggctggagctgcttcg

SEQ ID NO: 27 - DNA, 70 bp, Artificial Sequence
  Synthetic
gtttattgtgttttaaccacctgcccgtaaacctggagaaccatcgcgtgattccggggatccgtcgacc

SEQ ID NO: 28 - DNA, 70 bp, Artificial Sequence
  Synthetic
gctgggtagctccagcctgctttcctgcattacatcaccgcagcaaacgctgtaggctggagctgcttcg

SEQ ID NO: 29 - DNA, 55 bp, Artificial Sequence
  Synthetic
ggtcggcgcannscgtccgnnsacgnnsatgagcnnsgacggtccattcaacctg

SEQ ID NO: 30 - DNA, 55 bp, Artificial Sequence
  Synthetic
caggttgaatggaccgtcsnngctcatsnncgtsnncggacgsnntgcgccgacc

SEQ ID NO: 31 - DNA, 63 bp, Artificial Sequence
  Synthetic
ctgccgaaagtcggcnnsgtttatnnstacnnsaacgcannsgatcttccggctaaagcactg

SEQ ID NO: 32 - DNA, 63 bp, Artificial Sequence
  Synthetic
cagtgctttagccggaagatcsnntgcgttsnngtasnnataaacsnngccgactttcggcag

SEQ ID NO: 33 - DNA, 60 bp, Artificial Sequence
  Synthetic
ccgcaaaaagcgcgcnnsctgctgnnsctgnnsctgacgnnsaccaaagatccgcagcag

SEQ ID NO: 34 - DNA, 60 bp, Artificial Sequence
  Synthetic
ctgctgcggatctttggtsnncgtcagsnncagsnncagcagsnngcgcgctttttgcgg

SEQ ID NO: 35 - DNA, 42 bp, Artificial Sequence
  Synthetic
cgatcaaaccacctccccaggtggttttttcgtttacagggc

SEQ ID NO: 36 - DNA, 32 bp, Artificial Sequence
  Synthetic
ggtagtgaatatcgcgtcccaggacatgaacg

SEQ ID NO: 37 - DNA, 32 bp, Artificial Sequence
  Synthetic
ggtagtgaatatcgtgtcccaggacatgaacg

SEQ ID NO: 38 - DNA, 32 bp, Artificial Sequence
  Synthetic
ggtagtgaatatcctgtcccaggacatgaacg

SEQ ID NO: 39 - DNA, 37 bp, Artificial Sequence
  Synthetic
ggtgacgtcacggccatcaagcacggtgtcattcatc

SEQ ID NO: 40 - protein, 20 aa, Artificial Sequence
  Synthetic
MLPNITILATGGTIAGGGDS

SEQ ID NO: 41 - protein, 20 aa, Artificial Sequence
  Synthetic
GGTIAGGGDSATKSNYTVGK

SEQ ID NO: 42 - protein, 20 aa, Artificial Sequence
  Synthetic
ATKSNYTVGKVGVENLVNAV

SEQ ID NO: 43 - protein, 20 aa, Artificial Sequence
  Synthetic
VGVENLVNAVPQLKDIANVK

SEQ ID NO: 44 - protein, 20 aa, Artificial Sequence
  Synthetic
PQLKDIANVKGEQVVNIGSQ

SEQ ID NO: 45 - protein, 20 aa, Artificial Sequence
  Synthetic
GEQVVNIGSQDMNDNVWLTL

SEQ ID NO: 46 - protein, 20 aa, Artificial Sequence
  Synthetic
DMNDNVWLTLAKKINTDCDK

SEQ ID NO: 47 - protein, 20 aa, Artificial Sequence
  Synthetic
AKKINTDCDKTDGFVITHGT

SEQ ID NO: 48 - protein, 20 aa, Artificial Sequence
  Synthetic
TDGFVITHGTDTMEETAYFL

SEQ ID NO: 49 - protein, 20 aa, Artificial Sequence
  Synthetic
DTMEETAYFLDLTVKCDKPV

SEQ ID NO: 50 - protein, 20 aa, Artificial Sequence
  Synthetic
DLTVKCDKPVVMVGAMRPST

SEQ ID NO: 51 - protein, 20 aa, Artificial Sequence
  Synthetic
VMVGAMRPSTSMSADGPFNL

SEQ ID NO: 52 - protein, 20 aa, Artificial Sequence
  Synthetic
SMSADGPFNLYNAVVTAADK

SEQ ID NO: 53 - protein, 20 aa, Artificial Sequence
  Synthetic
YNAVVTAADKASANRGVLVV

SEQ ID NO: 54 - protein, 20 aa, Artificial Sequence
  Synthetic
ASANRGVLVVMNDTVLDGRD

SEQ ID NO: 55 - protein, 20 aa, Artificial Sequence
  Synthetic
MNDTVLDGRDVTKTNTTDVA

SEQ ID NO: 56 - protein, 20 aa, Artificial Sequence
  Synthetic
VTKTNTTDVATFKSVNYGPL

SEQ ID NO: 57 - protein, 20 aa, Artificial Sequence
  Synthetic
TFKSVNYGPLGYIHNGKIDY

SEQ ID NO: 58 - protein, 20 aa, Artificial Sequence
  Synthetic
GYIHNGKIDYQRTPARKHTS

SEQ ID NO: 59 - protein, 20 aa, Artificial Sequence
  Synthetic
QRTPARKHTSDTPFDVSKLN

SEQ ID NO: 60 - protein, 20 aa, Artificial Sequence
  Synthetic
DTPFDVSKLNELPKVGIVYN

SEQ ID NO: 61 - protein, 20 aa, Artificial Sequence
  Synthetic
ELPKVGIVYNYANASDLPAK

SEQ ID NO: 62 - protein, 19 aa, Artificial Sequence
  Synthetic
YANASDLPAKALVDAGYDG

SEQ ID NO: 63 - protein, 20 aa, Artificial Sequence
  Synthetic
ALVDAGYDGIVSAGVGNGNL

SEQ ID NO: 64 - protein, 20 aa, Artificial Sequence
  Synthetic
VSAGVGNGNLYKSVFDTLAT

SEQ ID NO: 65 - protein, 20 aa, Artificial Sequence
  Synthetic
YKSVFDTLATAAKTGTAVVR

SEQ ID NO: 66 - protein, 20 aa, Artificial Sequence
  Synthetic
AAKTGTAVVRSSRVPTGATT

SEQ ID NO: 67 - protein, 20 aa, Artificial Sequence
  Synthetic
SSRVPTGATTQDAEVDDAKY

SEQ ID NO: 68 - protein, 20 aa, Artificial Sequence
  Synthetic
QDAEVDDAKYGFVASGTLNP

SEQ ID NO: 69 - protein, 20 aa, Artificial Sequence
  Synthetic
GFVASGTLNPQKARVLLQLA

SEQ ID NO: 70 - protein, 20 aa, Artificial Sequence
  Synthetic
QKARVLLQLALTQTKDPQQI

SEQ ID NO: 71 - protein, 17 aa, Artificial Sequence
  Synthetic
LTQTKDPQQIQQIFNQY

SEQ ID NO: 72 - protein, 9 aa, Artificial Sequence
  Synthetic
IVYNYANAS

SEQ ID NO: 73 - protein, 9 aa, Artificial Sequence
  Synthetic
VLLQLALTQ

SEQ ID NO: 74 - protein, 9 aa, Artificial Sequence
  Synthetic
MRPSTSMSA

SEQ ID NO: 75 - protein, 9 aa, Artificial Sequence
  Synthetic
VRPPTRMSP

SEQ ID NO: 76 - protein, 9 aa, Artificial Sequence
  Synthetic
VVYGYANAS

SEQ ID NO: 77 - protein, 9 aa, Artificial Sequence
  Synthetic
VLLTLALTN

SEQ ID NO: 78 - protein, 10 aa, Artificial Sequence
  Synthetic
VLLQLALTYQ

SEQ ID NO: 79 - protein, 9 aa, Artificial Sequence
  Synthetic
XLLXLXLTX